(19) United States
(12) Patent Application Publication
(10) Pub. No.: US 2011/0104151 A1
(43) Pub. Date: May 5, 2011

(54) MICROPARTICLE COMPOSITIONS AND METHODS FOR TREATING AGE-RELATED MACULAR DEGENERATION

(76) Inventors: Heather Nettles, (US); Angela Stella, Pelham, AL (US)

(21) Appl. No.: 12/896,278

(22) Filed: Oct. 1, 2010

Related U.S. Application Data
(60) Provisional application No. 61/247,849, filed on Oct. 1, 2009.
(60) Provisional application No. 61/247,848, filed on Oct. 1, 2009.

(51) Int. Cl. A61K 39/395; A61P 27/02

(52) U.S. Cl. 424/133.1

(57) ABSTRACT

Disclosed herein are pharmaceutical compositions comprising microparticles that are useful for treating or preventing age-related macular degeneration. Also disclosed herein are microparticles that can be used to treat or prevent macular angiogenesis. Further disclosed are methods of making the microparticles and compositions and methods for treating or preventing macular degeneration and diseases, illnesses, or conditions relating to increased or abnormal macular angiogenesis.

### CROSS-REFERENCE TO RELATED APPLICATIONS

This application claims the benefit of priority of U.S. Provisional Application No. 61/247,849, filed Oct. 1, 2009, and U.S. Provisional Application No. 61/247,848, filed Oct. 1, 2009, each of which is incorporated herein by reference in its entirety.

### FIELD

Disclosed herein are pharmaceutical compositions useful for treating age-related macular degeneration. Also disclosed herein are microparticles that can deliver a pharmaceutically active ingredient useful as a treatment for preventing macular angiogenesis. Further disclosed are methods for treating macular degeneration and diseases, illnesses, or conditions relating to retinal edema and retinal neovascularization including increased or abnormal macular angiogenesis.

### BACKGROUND

Vascular endothelial growth factor A (VEGF-A) is one biological component that can trigger angiogenesis, which is the growth of new blood vessels. Various diseases, inter alia, ischemia, anemia, peripheral vascular disease, and atherosclerotic lesions can be treated by increasing angiogenesis. This is accomplished by stimulating the up-regulation of VEGF-A, thereby leading to increased blood circulation, hence increased oxygen supply, in the diseased tissue. In the eye, however, excessive vascularization can result in blood and fluid leaking into the eye. These leaky blood vessels can contribute to macular edema and choroidal neovascularization, resulting in the wet type of age-related macular degeneration (ARMD). The result of ARMD can be the loss of visual acuity or even blindness. Therefore, control of excessive macular vascularization is important in the treatment of macular degeneration. As such, it is a goal of medical professionals to provide a treatment for controlling or curing ARMD without inhibiting the beneficial effects of normal VEGF-A activity in the rest of the body.

Ranibizumab has been found to be an effective treatment of ARMD. Ranibizumab is a recombinant humanized IgG1 kappa isotype monoclonal antibody that inhibits VEGF activity by competitively binding to the receptor binding site of active forms of VEGF-A, including the biologically active, cleaved from of this molecule, VEGF₁₁₀. Hence, ranibizumab prevents binding of VEGF-A to its principle receptors VEGFR1 and VEGFR2 found on the surface of endothelial cells. This results in reduced endothelial cell proliferation, vascular leakage, and new blood vessel formation.

LUCENTIS™ is a medical formulation of ranibizumab and designed for intraocular injection directly into the vitreous humor of the eye, wherein the active ingredient ranibizumab penetrates the internal limiting membrane to access the subretinal space. These injections are typically given from 5 to 7 times a year to patients and in many instances are given monthly. Although a necessary manner of treatment, intraocular injections in general can lead to, inter alia, infection, retinal detachment, retinal disruption, cataracts, and bleeding, some of which can lead to blindness. As such, there is a need for compositions and methods for delivering ranibizumab to the eye that can reduce the frequency of injections, wherein an amount of ranibizumab is injected into the eye in a manner that slowly releases the drug and thus reduces the frequency of injections and the potential adverse side effects due to a high frequency of injections.

### SUMMARY

Disclosed herein are microparticles comprising ranibizumab wherein the microparticles can be used to deliver by intraocular injection directly into the vitreous humor of the eye a sufficient amount of ranibizumab for treating age-related macular degeneration such that the injections are only necessary at intervals of every 3 to 12 months.

One aspect of the disclosure relates to a pharmaceutical composition comprising a microparticle comprising:
a) from about 1 wt. % to about 15 wt. % ranibizumab; and
b) poly(D,L-lactide-co-glycolide) copolymer wherein the copolymer comprises:
   i) from about 75% to about 90% D,L-lactide units; and
   ii) from about 10% to about 25% glycolide units.

Another embodiment of this aspect relates to a pharmaceutical composition for treating age-related macular degeneration, comprising a microparticle comprising:
a) from about 1 wt. % to about 15 wt. % ranibizumab; and
b) poly(D,L-lactide-co-glycolide) copolymer wherein the copolymer comprises:
   i) from about 75% to about 90% D,L-lactide units; and
   ii) from about 10% to about 25% glycolide units;

wherein the microparticle is formed by a process comprising:
a) providing an aqueous phase comprising from about 1 wt. % to about 15 wt. % ranibizumab;
b) providing an organic phase comprising one or more poly(D,L-lactide-co-glycolide) copolymers wherein each copolymer comprises:
   i) from about 75% to about 90% D,L-lactide units; and
   ii) from about 10% to about 25% glycolide units;
c) combining the aqueous phase from step (a) with the organic phase from step (b) to form a primary emulsion;
d) combining the primary emulsion with an aqueous continuous phase to form a water/oil/water emulsion; and
e) combining the water/oil/water emulsion to an aqueous extraction phase and forming the microparticle.

A further embodiment of this aspect relates to a microparticle comprising ranibizumab in an amount of from 1 to 15 wt. % of the microparticle and a poly(lactide-co-glycolide) copolymer having from 75 wt. % to 90 wt. % lactide units and from 25 wt. % to 10 wt. % glycolide units, in the form of a microparticle.

Another aspect of the disclosure relates to a pharmaceutical composition comprising a microparticle comprising:
a) from about 1 wt. % to about 15 wt. % ranibizumab; and
b) poly(D,L-lactide).

Another embodiment of this aspect relates to a pharmaceutical composition for treating age-related macular degeneration, comprising a microparticle comprising:
a) from about 1 wt. % to about 15 wt. % ranibizumab; and
b) poly(D,L-lactide);

wherein the microparticle is formed by a process comprising:
a) providing an aqueous phase comprising from about 1 wt. % to about 15 wt. % ranibizumab;
b) providing an organic phase comprising poly(D,L-lactide);
c) combining the aqueous phase from step (a) with the organic phase from step (b) to form a primary emulsion;
d) combining the primary emulsion with an aqueous continuous phase to form a water/oil/water emulsion; and
e) combining the water/oil/water emulsion to an aqueous extraction phase and forming the microparticle

A further embodiment of this aspect relates to a microparticle comprising ranibizumab in an amount of from 1 to 15 wt. % of the microparticle and poly(D,L-lactide), in the form of a microparticle.

A further aspect of the disclosure relates to a pharmaceutical composition comprising a microparticle comprising:
a) from about 1 wt. % to about 15 wt. % ranibizumab; and
b) a polymer admixture comprising:
   i) from about 60% to about 99% poly(D,L-lactide); and
   ii) from about 1% to about 40% polycaprolactone.

Another embodiment of this aspect relates to a pharmaceutical composition for treating age-related macular degeneration, comprising a microparticle comprising:
a) from about 1 wt. % to about 15 wt. % ranibizumab; and
b) a polymer admixture comprising:
   i) from about 60% to about 99% poly(D,L-lactide); and
   ii) from about 1% to about 40% polycaprolactone; and

wherein the microparticle is formed by a process comprising:
a) providing an aqueous phase comprising from about 1 wt. % to about 15 wt. % ranibizumab;
b) providing an organic phase comprising a polymer admixture comprising:
   i) from about 60% to about 99% poly(D,L-lactide); and
   ii) from about 1% to about 40% polycaprolactone;
c) combining the aqueous phase from step (a) with the organic phase from step (b) to form a primary emulsion;
d) combining the primary emulsion with an aqueous continuous phase to form a water/oil/water emulsion; and
e) combining the water/oil/water emulsion to an aqueous extraction phase and forming the microparticle

A further embodiment of this aspect relates to microparticle comprising ranibizumab in an amount of from 1 to 15 wt. % of the microparticle and a polymer admixture comprising from about 60% to about 99% poly(D,L-lactide) and from about 1% to about 40% polycaprolactone, in the form of a microparticle.

A yet further aspect of the disclosure relates to a pharmaceutical composition comprising a microparticle comprising:
a) from about 1 wt. % to about 15 wt. % ranibizumab; and
b) an admixture of poly(D,L-lactide-co-glycolide) copolymers wherein the admixture comprises:
   i) from about 10 wt. % to about 90 wt. % of a first copolymer wherein the first copolymer comprises from about 65% to about 90% D,L-lactide units and from 10% to about 35% glycolide units; and
   ii) from about 10 wt. % to about 90 wt. % of a second copolymer wherein the second copolymer comprises from about 55% to about 90% D,L-lactide units and from 10% to about 45% glycolide units.

A further embodiment of this aspect relates to a pharmaceutical composition for treating age-related macular degeneration, comprising a microparticle comprising:
a) from about 1 wt. % to about 15 wt. % ranibizumab; and
b) an admixture of poly(D,L-lactide-co-glycolide) copolymers wherein the admixture comprises:
   i) from about 10% to about 90% of a first copolymer wherein the first copolymer comprises from about 65% to about 90% D,L-lactide units and from 10% to about 35% glycolide units; and
   ii) from about 10% to about 90% of a second copolymer wherein the second copolymer comprises from about 55% to about 90% D,L-lactide units and from 10% to about 45% glycolide units;

wherein the microparticle is formed by a process comprising:
a) providing an aqueous phase comprising from about 1 wt. % to about 15 wt. % ranibizumab;
b) providing an organic phase comprising an admixture of poly(D,L-lactide-co-glycolide) copolymers wherein the admixture comprises:
   i) from about 10% to about 90% of a first copolymer wherein the first copolymer comprises from about 65% to about 90% D,L-lactide units and from 10% to about 35% glycolide units; and
   ii) from about 10 wt. % to about 90 wt. % of a second copolymer wherein the second copolymer comprises from about 55% to about 90% D,L-lactide units and from 10% to about 45% glycolide units;
c) combining the aqueous phase from step (a) with the organic phase from step (b) to form a primary emulsion;
d) combining the primary emulsion with an aqueous continuous phase to form a water/oil/water emulsion; and
e) combining the water/oil/water emulsion to an aqueous extraction phase and forming the microparticle.

A still further embodiment of this aspect relates to a microparticle comprising ranibizumab in an amount of from 1 to 15 wt. % of the microparticle and an admixture of poly(D,L-lactide-co-glycolide) copolymers wherein the admixture comprises
a) from about 10% to about 90% of a first copolymer wherein the first copolymer comprises from about 65% to about 90% D,L-lactide units and from 10% to about 35% glycolide units; and
b) from about 10 wt. % to about 90 wt. % of a second copolymer wherein the second copolymer comprises from about 55% to about 90% D,L-lactide units and from 10% to about 45% glycolide units.

A yet further embodiment of this aspect relates to a method of preventing or treating age-related macular degeneration in a subject comprising administering to a subject in need of such treatment an effective amount of the disclosed compositions.

A still further embodiment of this aspect relates to a method of preventing or treating age-related macular degeneration in a subject comprising administering to a subject in need of such treatment an effective amount of the disclosed microparticles.

### BRIEF DESCRIPTION OF THE FIGURES

FIG. 1 depicts the in vitro elution profiles of ranibizumab microspheres in 100 mM PBS/0.5% BSA/0.05% Proclin 300 at 37° C. The line having solid triangles (▴) corresponds to a polymer blend of 8515 DLG 4.5E and 8515 DLG 6A; the line having clear triangles (Δ) corresponds to a polymer blend of 8515 DLG 5A and 7525 DLG 5.5E; the line having solid circles () corresponds to a polymer blend of 7525 DLG 7A and 6535 DLG 2E; the line having clear circles (◯) corresponds to a polymer blend of 7525 DLG 7E and 6535 DLG 4.5A; and the line having solid squares (□) corresponds to copolymer 8515 DLG 7A.

### DETAILED DESCRIPTION

The present invention can be understood more readily by reference to the following detailed description, examples, and claims, and their previous and following description. However, before the present compositions, articles, devices, and/or methods are disclosed and described, it is to be understood that this invention is not limited to the specific compositions, articles, devices, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its currently known embodiments. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

Before the present microparticles, copolymers, polymer admixtures, compounds, compositions, and/or methods are disclosed and described, it is to be understood that the aspects described herein are not limited to specific compounds, synthetic methods, or uses as such can, of course, vary. It is also to be understood that the terminology used herein is for the purpose of describing particular aspects only and, unless specifically defined herein, is not intended to be limiting.

In this specification and in the claims that follow, reference will be made to a number of terms that shall be defined to have the following meanings:

Throughout this specification, unless the context requires otherwise, the word “comprise,” or variations such as “comprises” or “comprising,” will be understood to imply the inclusion of a stated integer or step or group of integers or steps but not the exclusion of any other integer or step or group of integers or steps.

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, a “wt. %” or “weight percent” or “percent by weight” of a component, unless specifically stated to the contrary, refers to the ratio of the weight of the component to the total weight of the composition in which the component is included, expressed as a percentage.

By “contacting” is meant the physical contact of at least one substance to another substance.

By “sufficient amount” and “sufficient time” means an amount and time needed to achieve the desired result or results, e.g., dissolve a portion of the polymer.

“Admixture” or “blend” as generally used herein means a physical combination of two or more different components. In the case of polymers, an admixture, or blend, of polymers is a physical blend or combination of two or more different polymers as opposed to a copolymer which is single polymeric material that is comprised of two or more different monomers.

“Molecular weight” as used herein, unless otherwise specified, refers generally to the relative average molecular weight of the bulk polymer. In practice, molecular weight can be estimated or characterized in various ways including gel permeation chromatography (GPC) or capillary viscometry. GPC molecular weights are reported as the weight-average molecular weight (Mw) or as the number-average molecular weight (Mn). Capillary viscometry provides estimates of molecular weight as the Inherent Viscosity (IV) determined from a dilute polymer solution using a particular set of concentration, temperature, and solvent conditions. Unless otherwise specified, IV measurements are made at 30° C. on solutions prepared in chloroform at a polymer concentration of 0.5 g/dL.

“Controlled release” as used herein means the use of a material to regulate the release of another substance.

“Bioactive agent” is used herein to include ranibizumab in or on the disclosed microparticles.

“Excipient” is used herein to include any other compound or additive that can be contained in or on the microparticle that is not a therapeutically or biologically active compound. As such, an excipient should be pharmaceutically or biologically acceptable or relevant (for example, an excipient should generally be non-toxic to the subject). “Excipient” includes a single such compound and is also intended to include a plurality of excipients.

“Agent” is used herein to refer generally to compounds that are contained in or on a microparticle composition. Agent can include a bioactive agent or an excipient. “Agent” includes a single such compound and is also intended to include a plurality of such compounds

“Biocompatible” as used herein refers to a material that is generally non-toxic to the recipient and does not possess any significant untoward effects to the subject and, further, that any metabolites or degradation products of the material are non-toxic to the subject.

“Biodegradable” is generally referred to herein generally as a material that will erode to soluble species or that will degrade under physiologic conditions to smaller units or chemical species that are, themselves, non-toxic (biocompatible) to the subject and capable of being metabolized, eliminated, or excreted by the subject.

The term “microparticle” is used herein to include nanoparticles, microspheres, nanospheres, microcapsules, nanocapsules, and particles, in general. As such, the term microparticle refers to particles having a variety of internal structure and organizations including homogeneous matrices such as microspheres (and nanospheres) or heterogeneous core-shell matrices (such as microcapsules and nanocapsules), porous particles, multi-layer particles, among others. The term “microparticle” refers generally to particles that have sizes in the range of about 10 nanometers (nm) to about 2 mm (millimeters).

“Subject” is used herein to refer to any target of administration. The subject can be a vertebrate, for example, a mammal. Thus, the subject can be a human. The term does not denote a particular age or sex. Thus, adult and newborn subjects, as well as fetuses, whether male or female, are intended to be covered. A “patient” refers to a subject afflicted with a disease or disorder and includes human and veterinary subjects.

Disclosed are compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a number of different polymers and agents are disclosed and discussed, each and every combination and permutation of the polymer and agent are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

In a first major aspect, the disclosed microparticles relate to microparticles that comprise:
a) from about 1 wt. % to about 15 wt. % ranibizumab; and
b) a poly(D,L-lactide-co-glycolide) copolymer comprising:
   i) from about 75% to about 90% D,L-lactide units; and
   ii) from about 10% to about 25% glycolide units.

The disclosed microparticles of this aspect comprise from about 1 wt. % to about 15 wt. % ranibizumab. In one embodiment of this aspect, the microparticles comprise from about 5 wt. % to about 10 wt. % of ranibizumab. In another embodiment, the microparticles comprise from about 6 wt. % to about 9 wt. % of ranibizumab. In a further embodiment, the microparticles comprise from about 7 wt. % to about 9 wt. % of ranibizumab. One example comprises 8 wt. % of ranibizumab. The microparticles, however, can comprise any amount of ranibizumab from about 1 wt. % to about 15 wt. %, for example, 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, and 15 wt. %. Included in these amounts are fractional amounts of ranibizumab, for example, 1.1 wt. %, 2.75 wt. %, 4.33 wt. %, and the like.

The disclosed microparticles of this aspect comprise a copolymer having from about 75% to about 90% D,L-lactide units and the balance glycolide units. In one embodiment, the microparticles can comprise from about 80% to about 90% D,L-lactide units. In another embodiment, the microparticles can comprise from about 82% to about 88% D,L-lactide units. In a further embodiment, the microparticles can comprise from about 78% to about 88% D,L-lactide units. The microparticles, however, can comprise any amount of D,L-lactide units from about 75% to about 90 wt. %, for example, 75%, 76%, 77%, 78%, 79%, 80%, 81%, 82%, 83%, 84%, 85%, 86%, 87%, 88%, 89%, and 90%. Included in these amounts are fractional amounts of ranibizumab, for example, 84.5%, 86.2%, 87.75%, and the like. The balance of the copolymer composition is glycolide units.

The disclosed microparticles of this aspect comprise poly(D,L-lactide-co-glycolide) copolymer having an intrinsic viscosity (IV) of from about 0.2 to about 0.8 dL/g. In one embodiment, the copolymer has an IV of from about 0.3 to about 0.8 dL/g. In another embodiment, the copolymer has an IV of from about 0.4 to about 0.8 dL/g. In a further embodiment, the copolymer has an IV of from about 0.4 to about 0.7 dL/g. The microparticles, however, can comprise any IV from about 0.2 to about 0.8 dL/g, for example, 0.2 dL/g, 0.25 dL/g, 0.3 dL/g, 0.35 dL/g, 0.4 dL/g, 0.45 dL/g, 0.5 dL/g, 0.55 dL/g, 0.6 dL/g, 0.65 dL/g, 0.7 dL/g, 0.75 dL/g, 0.8 dL/g, and the like, or any fraction amount thereof, for example, 0.557 dL/g. Alternatively, the formulator can express the inherent viscosity in cm³/g if convenient.

The disclosed microparticles of this aspect have an average or mean particle size of from about 20 microns to about 125 microns. In one embodiment the range of mean particle size is from about 40 microns to about 90 microns. In another embodiment the range of mean particle sizes is from about 50 microns to about 80 microns. Particle size distributions are measured by laser diffraction techniques known to those of skill in the art.

In a further embodiment of this aspect, the bulk drug product that is used during preparation of the microparticles can comprise one or more water soluble carriers or excipients. Such carriers or excipients may generally include sugars, saccharides, polysaccharides, surfactants, buffer salts, bulking agents, and the like. A non-limiting example of an excipient is 2-(hydroxy-methyl)-6-[3,4,5-trihydroxy-6-(hydroxymethyl)tetrahydropyran-2-yl]oxy-tetrahydropyran-3,4,5-triol, “trehalose.” One embodiment of the disclosed process includes a bulk drug product used during preparation of the microparticles comprising 1 wt. % to 200 wt. % trehalose based on the weight of drug in the starting bulk drug product. In a further embodiment, the bulk drug product used during preparation of the microparticles comprises about 10 wt. % to 50 wt. % trehalose based on the weight of drug in the starting bulk drug product. In a non-limiting example of this aspect the bulk drug product comprises 25 wt % to 35 wt % trehalose. Another non-limiting example of an excipient is the surfactant polysorbate 20 (or Tween 20). One embodiment of the disclosed process includes a bulk drug product used during preparation of the microparticles comprising 0.01 wt % to 5 wt % polysorbate 20 based on the weight of drug in the starting bulk drug product. In a yet further embodiment, the bulk drug product used during preparation of the microparticles comprises about 0.05 wt % to 0.25 wt % polysorbate 20 based on the weight of drug in the starting bulk drug product. In a non-limiting example of this aspect the bulk drug product comprises about 0.1 wt % polysorbate 20. Further, the bulk drug product may contain two or more such carriers or excipients. A non-limiting example includes a bulk drug product comprising 25 wt. % to 35 wt. % trehalose and about 0.1 wt. % polysorbate 20 based on the weight of drug in the starting bulk drug product.

Disclosed herein are compositions that can be used for treating age related macular degeneration. One aspect relates to compositions comprising:

A) a microparticle comprising:
a) from about 1 wt. % to about 15 wt. % ranibizumab; and
b) a poly(D,L-lactide-co-glycolide) copolymer comprising:
   i) from about 75% to about 90% D,L-lactide units; and
   ii) from about 10% to about 25% glycolide units; and

B) a pharmaceutically acceptable carrier.

In one embodiment, the composition comprises:

A) a microparticle comprising:
a) from about 5 wt. % to about 10 wt. % ranibizumab; and
b) a poly(D,L-lactide-co-glycolide) copolymer wherein the copolymer comprises:
   i) 85% D,L-lactide units; and
   ii) 15% glycolide units; and

B) a pharmaceutically acceptable carrier.

One example of this embodiment relates to a compositions comprising:

A) a microparticle comprising:
a) from about 5 wt. % to about 10 wt. % ranibizumab; and
b) a poly(D,L-lactide-co-glycolide) copolymer wherein the copolymer comprises:
   i) 85% D,L-lactide units; and
   ii) 15% glycolide units;
wherein the copolymer has a intrinsic viscosity of from about 0.45 dL/g to about 0.70 dL/g, or from about 0.45 dL/g to about 0.60 dL/g, or from about 0.60 dL/g to about 0.70 dL/g; and

B) a pharmaceutically acceptable carrier.

A further aspect relates to a pharmaceutical composition comprising:

A) from about 1 mg to about 500 mg of a microparticle comprising:
a) from about 1 wt. % to about 15 wt. % ranibizumab; and
b) one or more poly(D,L-lactide-co-glycolide) copolymers wherein the copolymers comprise:
   i) from about 75% to about 90% D,L-lactide units; and
   ii) from about 10% to about 25% glycolide units

B) a pharmaceutically acceptable carrier;

wherein the composition contains from about 1 wt. % to about 50 wt. % of solids.

The disclosed microparticles of this aspect can be prepared according to any microparticle preparation process. In one aspect, the microparticles are prepared according to U.S. Pat. No. 5,407,609 included herein by reference in its entirety. In another aspect, the process comprises:
a) providing an aqueous phase comprising from about 1 wt. % to about 50 wt. % ranibizumab;
b) providing an organic phase comprising one or more poly(D,L-lactide-co-glycolide) copolymers wherein each copolymer comprises:
   i) from about 75% to about 90% D,L-lactide units; and
   ii) from about 10% to about 25% glycolide units;
c) combining the aqueous phase from step (a) with the organic phase from step (b) to form a primary emulsion;
d) combining the primary emulsion with an aqueous continuous phase to form a water/oil/water emulsion; and
e) combining the water/oil/water emulsion with an aqueous extraction phase and forming the microparticle.

The disclosed process for forming the microparticles of this aspect can use in step (b) any non-halogenated hydrocarbon organic solvent or any halogenated organic solvent. In addition, a solvent can be added to the aqueous continuous phase of step (c) in an amount up to the point of saturation.

By “non-halogenated organic solvent” is meant an organic solvent suitable for serving as a primary solvent for dissolving the polymers disclosed herein, for example, in step (b). Non-limiting examples of non-halogenated solvents include: ketones, inter alia, acetone, methyl ethyl ketone; alcohols, inter alia, methanol, ethanol, n-propanol, iso-propanol, benzyl alcohol, glycerol; ethers, inter alia, diethyl ether, tetrahydrofuran, glyme, diglyme; esters, inter alia, methyl acetate, ethyl acetate; hydrocarbons, inter alia, n-pentane, iso-pentane, hexane, heptane, isooctane, benzene, toluene, xylene (all isomers); polar aprotic solvents, inter alia, dimethyl formamide, dimethyl acetamide, dimethyl sulfoxide, hexamethylphosphoramide, and the like.

By “halogenated solvents” is meant halogenated organic solvents, i.e., C₁-C₄ halogenated alkanes, non-limiting examples of which include carbon tetrachloride, chloroform, methylene chloride, chloroethane, 1,1-dichloroethane, 1,1,1-trichloroethane, and 1,2-dichloroethane.

In one aspect of the process for forming the disclosed microparticles of this aspect, ethyl acetate is used to dissolve or disperse the poly(D,L-lactide-co-glycolide) copolymer in step (b). In another aspect of the process for forming the disclosed microparticles of this aspect, methylene chloride is used to dissolve or disperse the poly(D,L-lactide-co-glycolide) copolymer in step (b).

In another embodiment of the process for forming the disclosed microparticles of this aspect, a salt can be added to the aqueous continuous phase of step (c) in an amount up to the point of saturation. A salt can be added alone or in combination with an organic solvent added to the continuous phase. In one aspect, a salt is present in an amount from about 0.1 M to about 10 M. In another aspect, a salt is present in an amount from about 0.5 M to about 5 M. However, the concentration of the salt in the continuous phase can be any amount from about 0.1 M to about 10 M. One example of a salt that can be used in the continuous phase is sodium chloride, for example, 1.5M NaCl, 2 M NaCl, 2.5 M NaCl, and the like. In one aspect of this embodiment, the aqueous continuous phase can comprise 2 M NaCl. A non-limiting example of the disclosed process for forming microparticles of this aspect includes the following step (d):
d) combining the primary emulsion (water in oil emulsion formed in step (c)) with an aqueous continuous phase comprising 2 M NaCl to form a water/oil/water emulsion.

In a yet further embodiment of the process for forming the disclosed microparticles of this aspect, a surfactant, for example, poly(vinyl alcohol) (PVA), can be added to the aqueous solution comprising ranibizumab of step (a), the continuous phase of step (c), or to both. The amount of PVA can be from about 0.05 wt. % to about 1 wt. % of the aqueous solution of step (a). The amount of surfactant can be from about 0.5 wt. % to about 3 wt. % of the continuous phase. In one aspect of this embodiment, the aqueous phase of step (a) comprises from about 0.1 wt. % to about 0.5 wt. % of poly(vinyl alcohol). In another aspect of this embodiment, the continuous phase comprises from about 1.5 wt. % to about 2.5 wt. % of poly(vinyl alcohol).

The aqueous Continuous Phase solution can comprise one or more other surfactants or emulsifiers. Other surfactants and emulsifying agents include most any physiologically acceptable emulsifiers. Examples include lecithin such as egg lecithin or soya bean lecithin or synthetic lecithins. Emulsifiers also include surfactants such as free fatty acids, esters of fatty acids of polyoxyalkylene compounds like polyoxpropylene glycol and polyoxyethylene glycol; ethers of fatty alcohols with polyoxyalkylene glycols; esters of fatty acids with polyoxyalkylated sorbitan; soaps; glycerol-polyalkylene stearate; glycerol-polyoxyethylene ricinoleate; homo- and co-polymers of polyalkylene glycols; polyethoxylated soya-oil and castor oil as well as hydrogenated derivatives; ethers and esters of sucrose or other carbohydrates with fatty acids, fatty alcohols, these being optionally polyoxyalkylated; mono-, di-, and tri-glycerides of saturated or unsaturated fatty acids, glycerides or soya-oil and sucrose. Other emulsifiers include natural and synthetic forms of bile salts or bile acids, both conjugated with amino acids and unconjugated such as taurodeoxycholate, and cholic acid.

One embodiment of the process for preparing the microparticles of this aspect relates to the use of both a surfactant, for example, poly(vinyl alcohol) and a salt, for example, NaCl in the aqueous continuous phase of step (c). In one aspect, the amount of poly(vinyl alcohol) can be from about 0.5 wt. % to about 3.5 wt. % when a salt is present. In another aspect, the amount of poly(vinyl alcohol) can be about 2 wt. % of the continuous phase when a salt is present.

In one embodiment of the process, the aqueous continuous phase of step (c) can comprise poly(vinyl alcohol) and salt and, optionally, the organic solvent used in the preparation of the dispersed phase solution in quantities up to saturating levels in the continuous phase solution.

The solvent phase of step (b) comprising poly(D,L-lactide-co-glycolide) copolymer can comprise from about 10 wt. % to about 30 wt. % of copolymer. In one aspect of this embodiment, the solvent phase can comprise from about 15 wt. % to about 25 wt. % of copolymer. A non-limiting example of the disclosed process for forming microparticles of this aspect includes the following step (b):
b) providing an organic phase comprising about 20 wt. % of one or more poly(D,L-lactide-co-glycolide) copolymers wherein each copolymer comprises:
   i) from about 75% to about 90% D,L-lactide units; and
   ii) from about 10% to about 25% glycolide units.

One embodiment of the disclosed process for forming a microparticle of this aspect includes a process comprising:
a) providing an aqueous phase comprising from about 10 wt. % to about 30 wt. % ranibizumab and from about 0.1 wt. % to about 0.5 wt. % of poly(vinyl alcohol);
b) providing an organic phase comprising ethyl acetate and from about 10 wt. % to about 30 wt. % of one or more poly(D,L-lactide-co-glycolide) copolymers having an intrinsic viscosity of from about 0.2 dL/g to about 0.8 dL/g, wherein the copolymers comprise:
   i) from about 75% to about 90% D,L-lactide units; and
   ii) from about 10% to about 25% glycolide units;
c) combining the aqueous phase from step (a) with the organic phase from step (b) to form a primary emulsion;
d) combining the primary emulsion with an aqueous continuous phase comprising from about 1.5 wt. % to about 2.5 wt. % of poly(vinyl alcohol) to form a water/oil/water emulsion; and
e) combining the water/oil/water emulsion with an aqueous extraction phase and forming the microparticle.

Another embodiment of the disclosed process for forming a microparticle of this aspect includes a process comprising:
a) providing an aqueous phase comprising from about 10 wt. % to about 30 wt. % ranibizumab and from about 0.1 wt. % to about 0.5 wt. % of poly(vinyl alcohol);
b) providing an organic phase comprising ethyl acetate and from about 10 wt. % to about 30 wt. % of one or more poly(D,L-lactide-co-glycolide) copolymers having an intrinsic viscosity of from about 0.2 dL/g to about 0.8 dL/g, wherein the copolymers comprise:
   i) from about 75% to about 90% D,L-lactide units; and
   ii) from about 10% to about 25% glycolide units;
c) combining the aqueous phase from step (a) with the organic phase from step (b) to form a primary emulsion;
d) combining the primary emulsion with an aqueous continuous phase comprising ethyl acetate and from about 1.5 wt. % to about 2.5 wt. % of poly(vinyl alcohol) to form a water/oil/water emulsion; and
e) combining the water/oil/water emulsion with an aqueous extraction phase and forming the microparticle.

A further non-limiting example of the disclosed process for forming a microparticle of this aspect includes a process comprising:
a) providing an aqueous phase comprising from about 10 wt. % to about 30 wt. % ranibizumab and from about 0.1 wt. % to about 0.5 wt. % of poly(vinyl alcohol);
b) providing an organic phase comprising methylene chloride and from about 10 wt. % to about 30 wt. % of one or more poly(D,L-lactide-co-glycolide) copolymers having an intrinsic viscosity of from about 0.2 dL/g to about 0.8 dL/g, wherein the copolymers comprise:
   i) from about 75% to about 90% D,L-lactide units; and
   ii) from about 10% to about 25% glycolide units;
c) combining the aqueous phase from step (a) with the organic phase from step (b) to form a primary emulsion;
d) combining the primary emulsion with an aqueous continuous phase comprising from about 1.5 wt. % to about 2.5 wt. % of poly(vinyl alcohol) to form a water/oil/water emulsion; and
e) combining the water/oil/water emulsion with an aqueous extraction phase and forming the microparticle.

Yet another embodiment of the disclosed process for forming a microparticle of this aspect includes a process comprising:
a) providing an aqueous phase comprising from about 10 wt. % to about 30 wt. % ranibizumab and from about 0.1 wt. % to about 0.5 wt. % of poly(vinyl alcohol);
b) providing an organic phase comprising methylene chloride and from about 10 wt. % to about 30 wt. % of one or more poly(D,L-lactide-co-glycolide) copolymers having an intrinsic viscosity of from about 0.2 dL/g to about 0.8 dL/g, wherein the copolymers comprise:
   i) from about 75% to about 90% D,L-lactide units; and
   ii) from about 10% to about 25% glycolide units;
c) combining the aqueous phase from step (a) with the organic phase from step (b) to form a primary emulsion;
d) combining the primary emulsion with an aqueous continuous phase comprising methylene chloride and from about 1.5 wt. % to about 2.5 wt. % of poly(vinyl alcohol) to form a water/oil/water emulsion; and
e) combining the water/oil/water emulsion with an aqueous extraction phase and forming the microparticle.

A further non-limiting example of the disclosed process for forming a microparticle of this aspect includes a process comprising:
a) providing an aqueous phase comprising from about 10 wt. % to about 30 wt. % ranibizumab;
b) providing an organic phase comprising ethyl acetate and from about 10 wt. % to about 30 wt. % of one or more poly(D,L-lactide-co-glycolide) copolymer having an intrinsic viscosity of from about 0.2 dL/g to about 0.8 dL/g, wherein the copolymers comprise:
   i) from about 75% to about 90% D,L-lactide units; and
   ii) from about 10% to about 25% glycolide units;
c) combining the aqueous phase from step (a) with the organic phase from step (b) to form a primary emulsion;
d) combining the primary emulsion with an aqueous continuous phase comprising from about 1.5 M to about 2.5 M NaCl to form a water/oil/water emulsion; and
e) combining the water/oil/water emulsion with an aqueous extraction phase and forming the microparticle.

A yet further non-limiting example of the disclosed process for forming a microparticle of this aspect includes a process comprising:
a) providing an aqueous phase comprising from about 10 wt. % to about 30 wt. % ranibizumab;
b) providing an organic phase comprising methylene chloride and from about 10 wt. % to about 30 wt. % of one or more poly(D,L-lactide-co-glycolide) copolymer having an intrinsic viscosity of from about 0.2 dL/g to about 0.8 dL/g, wherein the copolymers comprise:
   i) from about 75% to about 90% D,L-lactide units; and
   ii) from about 10% to about 25% glycolide units;
c) combining the aqueous phase from step (a) with the organic phase from step (b) to form a primary emulsion;
d) combining the primary emulsion with an aqueous continuous phase comprising from about 1.5 M to about 2.5 M NaCl to form a water/oil/water emulsion; and
e) combining the water/oil/water emulsion with an aqueous extraction phase and forming the microparticle.

A still further non-limiting example of the disclosed process for forming a microparticle of this aspect includes a process comprising:
a) providing an aqueous phase comprising from about 10 wt. % to about 30 wt. % ranibizumab and from about 0.1 wt. % to about 0.5 wt. % of poly(vinyl alcohol);
b) providing an organic phase comprising ethyl acetate and from about 10 wt. % to about 30 wt. % of one or more poly(D,L-lactide-co-glycolide) copolymer having an intrinsic viscosity of from about 0.2 dL/g to about 0.8 dL/g, wherein the copolymers comprise:
   i) from about 75% to about 90% D,L-lactide units; and
   ii) from about 10% to about 25% glycolide units;
c) combining the aqueous phase from step (a) with the organic phase from step (b) to form a primary emulsion;
d) combining the primary emulsion with an aqueous continuous phase comprising from about 1.5 M to about 2.5 M NaCl and from about 1.5 wt. % to about 2.5 wt. % poly(vinyl alcohol) to form a water/oil/water emulsion; and
e) combining the water/oil/water emulsion with an aqueous extraction phase and forming the microparticle.

A still yet further non-limiting example of the disclosed process for forming a microparticle of this aspect includes a process comprising:
a) providing an aqueous phase comprising from about 10 wt. % to about 30 wt. % ranibizumab and from about 0.1 wt. % to about 0.5 wt. % of poly(vinyl alcohol);
b) providing an organic phase comprising methylene chloride and from about 10 wt. % to about 30 wt. % of one or more poly(D,L-lactide-co-glycolide) copolymers having an intrinsic viscosity of from about 0.2 dL/g to about 0.8 dL/g, wherein the copolymers comprise:
   i) from about 75% to about 90% D,L-lactide units; and
   ii) from about 10% to about 25% glycolide units;
c) combining the aqueous phase from step (a) with the organic phase from step (b) to form a primary emulsion;
d) combining the primary emulsion with an aqueous continuous phase comprising from about 1.5 M to about 2.5 M NaCl and from about 1.5 wt. % to about 2.5 wt. % poly(vinyl alcohol) to form a water/oil/water emulsion; and
e) combining the water/oil/water emulsion with an aqueous extraction phase and forming the microparticle.

Disclosed herein are methods for treating or preventing age related macular degeneration. Further disclosed are methods for treating macular degeneration and diseases, illnesses, or conditions relating to retinal edema and retinal neovascularization including increased or abnormal macular angiogenesis.

The first aspect relates to administering to a subject in need of such treatment with a microparticle comprising:
a) from about 1 wt. % to about 15 wt. % ranibizumab; and
b) poly(D,L-lactide-co-glycolide) copolymer wherein the copolymer comprises:
   i) from about 75% to about 90% D,L-lactide units; and
   ii) from about 10% to about 25% glycolide units.

One embodiment of this aspect relates to a method for treating or preventing age related macular degeneration comprising, administering to a subject in need of treatment a composition comprising:

A) a microparticle comprising:
a) from about 1 wt. % to about 15 wt. % ranibizumab; and
b) a poly(D,L-lactide-co-glycolide) copolymer comprising:
   i) from about 75% to about 90% D,L-lactide units; and
   ii) from about 10% to about 25% glycolide units; and

B) a pharmaceutically acceptable carrier.

A non-limiting example of this embodiment relates to a method for treating or preventing age related macular degeneration comprising, administering to a subject in need of treatment a composition comprising:

A) a microparticle comprising:
a) about 8 wt. % ranibizumab; and
b) poly(D,L-lactide-co-glycolide) comprising:
   i) about 85% D,L-lactide units; and
   ii) about 15% glycolide units; and

B) a pharmaceutically acceptable carrier.

A further aspect relates to administering to a subject in need of treatment with a pharmaceutical composition comprising:

A) from about 1 mg to about 500 mg of a microparticle comprising:
a) from about 1 wt. % to about 15 wt. % ranibizumab; and
b) a poly(D,L-lactide-co-glycolide) copolymer comprising:
   i) from about 75% to about 90% D,L-lactide units; and
   ii) from about 10% to about 25% glycolide units

B) a pharmaceutically acceptable carrier;

wherein the composition contains from about 1 wt. % to about 50 wt. % of solids.

In a second major aspect, the disclosed microparticles relate to microparticles that comprise:
a) from about 1 wt. % to about 15 wt. % ranibizumab; and
b) poly(D,L-lactide).

The disclosed microparticles of this aspect comprise from about 1 wt. % to about 15 wt. % ranibizumab. In one embodiment of this aspect, the microparticles comprise from about 5 wt. % to about 10 wt. % of ranibizumab. In another embodiment, the microparticles comprise from about 2 wt. % to about 7 wt. % of ranibizumab. In a further embodiment, the microparticles comprise from about 4 wt. % to about 8 wt. % of ranibizumab. One example comprises from about 5 wt. % to about 6 wt. % of ranibizumab. The microparticles, however, can comprise any amount of ranibizumab from about 1 wt. % to about 15 wt. %, for example, 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, and 15 wt. %. Included in these amounts are fractional amounts of ranibizumab, for example, 1.1 wt. %, 2.75 wt. %, 4.33 wt. %, and the like.

The disclosed microparticles of this aspect comprise poly(D,L-lactide) having an intrinsic viscosity (IV) of from about 0.2 to about 0.8 dL/g. In one embodiment, the copolymer has an IV of from about 0.3 to about 0.8 dL/g. In another embodiment, the copolymer has an IV of from about 0.4 to about 0.8 dL/g. In a further embodiment, the copolymer has an IV of from about 0.3 to about 0.5 dL/g. The microparticles, however, can comprise any IV from about 0.2 to about 0.8 dL/g, for example, 0.2 dL/g, 0.25 dL/g, 0.3 dL/g, 0.35 dL/g, 0.4 dL/g, 0.45 dL/g, 0.5 dL/g, 0.55 dL/g, 0.6 dL/g, 0.65 dL/g, 0.7 dL/g, 0.75 dL/g, 0.8 dL/g, and the like, or any fraction amount thereof, for example, 0.557 dL/g. Alternatively, the formulator can express the inherent viscosity in cm³/g if convenient.

The disclosed microparticles of this aspect have an average or mean particle size of from about 20 microns to about 125 microns. In one embodiment the range of mean particle size is from about 40 microns to about 90 microns. In another embodiment the range of mean particle sizes is from about 50 microns to about 80 microns. Particle size distributions are measured by laser diffraction techniques known to those of skill in the art.

In a further embodiment of this aspect, the bulk drug product that is used during preparation of the microparticles can comprise one or more water soluble carriers or excipients. Such carriers or excipients may generally include sugars, saccharides, polysaccharides, surfactants, buffer salts, bulking agents, and the like. A non-limiting example of an excipient is 2-(hydroxy-methyl)-6-[3,4,5-trihydroxy-6-(hydroxymethyl)tetrahydropyran-2-yl]oxy-tetrahydropyran-3,4,5-triol, “trehalose.” One embodiment of the disclosed process includes a bulk drug product used during preparation of the microparticles comprising 1 wt % to 200 wt % trehalose based on the weight of drug in the starting bulk drug product. In a further embodiment, the bulk drug product used during preparation of the microparticles comprises about 10 wt. % to 50 wt. % trehalose based on the weight of drug in the starting bulk drug product. In a non-limiting example of this aspect the bulk drug product comprises 25 wt % to 35 wt % trehalose. Another non-limiting example of an excipient is the surfactant polysorbate 20 (or Tween 20). One embodiment of the disclosed process includes a bulk drug product used during preparation of the microparticles comprising 0.01 wt % to 5 wt % polysorbate 20 based on the weight of drug in the starting bulk drug product. In a yet further embodiment, the bulk drug product used during preparation of the microparticles comprises about 0.05 wt % to 0.25 wt % polysorbate 20 based on the weight of drug in the starting bulk drug product. In a non-limiting example of this aspect the bulk drug product comprises about 0.1 wt % polysorbate 20. Further, the bulk drug product may contain two or more such carriers or excipients. A non-limiting example includes a bulk drug product comprising 25 wt % to 35 wt % trehalose and about 0.1 wt % polysorbate 20 based on the weight of drug in the starting bulk drug product.

Another aspect of the disclosed compositions relates to compositions comprising:

A) a microparticle comprising:
a) from about 1 wt. % to about 15 wt. % ranibizumab; and
b) poly(D,L-lactide); and

B) a pharmaceutically acceptable carrier.

In one embodiment, the composition comprises:

A) a microparticle comprising:
a) from about 1 wt. % to about 15 wt. % ranibizumab; and
b) poly(D,L-lactide);
   wherein the poly(D,L-lactide) has an intrinsic viscosity of from about 0.2 to about 0.8 dL/g; and

B) a pharmaceutically acceptable carrier.

A further aspect relates to a pharmaceutical composition comprising:

A) from about 1 mg to about 500 mg of a microparticle comprising:
a) from about 1 wt. % to about 15 wt. % ranibizumab; and
b) poly(D,L-lactide); and
c) a pharmaceutically acceptable carrier;

wherein the composition contains from about 1 wt. % to about 50 wt. % of solids.

The disclosed microparticles of this aspect can be prepared according to any microparticle preparation process. In one aspect, the microparticles are prepared according to U.S. Pat. No. 5,407,609 included herein by reference in its entirety. In another aspect, the process comprises:
a) providing an aqueous phase comprising from about 1 wt. % to about 50 wt. % ranibizumab;
b) providing an organic phase comprising poly(D,L-lactide);
c) combining the aqueous phase from step (a) with the organic phase from step (b) to form a primary emulsion;
d) combining the primary emulsion with an aqueous continuous phase to form a water/oil/water emulsion; and
e) combining the water/oil/water emulsion with an aqueous extraction phase and forming the microparticle.

The disclosed process for forming the microparticles of this aspect can use in step (b) any non-halogenated hydrocarbon organic solvent or any halogenated organic solvent. In addition, a solvent can be added to the aqueous continuous phase of step (c) in an amount up to the point of saturation.

By “non-halogenated organic solvent” is meant an organic solvent suitable for serving as a primary solvent for dissolving the polymers disclosed herein, for example, in step (b). Non-limiting examples of non-halogenated solvents include: ketones, inter alia, acetone, methyl ethyl ketone; alcohols, inter alia, methanol, ethanol, n-propanol, iso-propanol, benzyl alcohol, glycerol; ethers, inter alia, diethyl ether, tetrahydrofuran, glyme, diglyme; esters, inter alia, methyl acetate, ethyl acetate; hydrocarbons, inter alia, n-pentane, iso-pentane, hexane, heptane, isooctane, benzene, toluene, xylene (all isomers); polar aprotic solvents, inter alia, dimethyl formamide, dimethyl acetamide, dimethyl sulfoxide, hexamethylphosphoramide, and the like.

By “halogenated solvents” is meant halogenated organic solvents, i.e., C₁-C₄ halogenated alkanes, non-limiting examples of which include carbon tetrachloride, chloroform, methylene chloride, chloroethane, 1,1-dichloroethane, 1,1,1-trichloroethane, and 1,2-dichloroethane.

In one aspect of the process for forming the disclosed microparticles of this aspect, ethyl acetate is used to dissolve or disperse the poly(D,L-lactide) copolymer in step (b). In another aspect of the process for forming the disclosed microparticles of this aspect, methylene chloride is used to dissolve the poly(D,L-lactide) copolymer in step (b).

In another embodiment of the process for forming the disclosed microparticles of this aspect, a salt can be added to the aqueous continuous phase of step (c) in an amount up to the point of saturation. A salt can be added alone or in combination with an organic solvent added to the continuous phase. In one aspect, a salt is present in an amount from about 0.1 M to about 10 M. In another aspect, a salt is present in an amount from about 0.5 M to about 5 M. However, the concentration of the salt in the continuous phase can be any amount from about 0.1 M to about 10 M. One example of a salt that can be used in the continuous phase is sodium chloride, for example, 1.5M NaCl, 2 M NaCl, 2.5 M NaCl, and the like. In one aspect of this embodiment, the aqueous continuous phase can comprise 2 M

NaCl. A non-limiting example of the disclosed process for forming microparticles of this aspect includes the following step (d):
d) combining the primary emulsion (water in oil emulsion formed in step (c)) with an aqueous continuous phase comprising 2 M NaCl to form a water/oil/water emulsion.

In a yet further embodiment of the process for forming the disclosed microparticles of this aspect, a surfactant, for example, poly(vinyl alcohol) (PVA), can be added to the aqueous solution comprising ranibizumab of step (a), the continuous phase of step (c), or to both. The amount of PVA can be from about 0.05 wt. % to about 1 wt. % of the aqueous solution of step (a). The amount of surfactant can be from about 0.5 wt. % to about 3 wt. % of the continuous phase. In one aspect of this embodiment, the aqueous phase of step (a) comprises from about 0.1 wt. % to about 0.5 wt. % of poly(vinyl alcohol). In another aspect of this embodiment, the continuous phase comprises from about 1.5 wt. % to about 2.5 wt. % of poly(vinyl alcohol).

The aqueous Continuous Phase solution can comprise one or more other surfactants or emulsifiers. Other surfactants and emulsifying agents include most any physiologically acceptable emulsifiers. Examples include lecithin such as egg lecithin or soya bean lecithin or synthetic lecithins. Emulsifiers also include surfactants such as free fatty acids, esters of fatty acids of polyoxyalkylene compounds like polyoxpropylene glycol and polyoxyethylene glycol; ethers of fatty alcohols with polyoxyalkylene glycols; esters of fatty acids with polyoxyalkylated sorbitan; soaps; glycerol-polyalkylene stearate; glycerol-polyoxyethylene ricinoleate; homo- and co-polymers of polyalkylene glycols; polyethoxylated soya-oil and castor oil as well as hydrogenated derivatives; ethers and esters of sucrose or other carbohydrates with fatty acids, fatty alcohols, these being optionally polyoxyalkylated; mono-, di-, and tri-glycerides of saturated or unsaturated fatty acids, glycerides or soya-oil and sucrose. Other emulsifiers include natural and synthetic forms of bile salts or bile acids, both conjugated with amino acids and unconjugated such as taurodeoxycholate, and cholic acid.

One embodiment of the process for preparing the microparticles of this aspect relates to the use of both a surfactant, for example, poly(vinyl alcohol) and a salt, for example, NaCl in the aqueous continuous phase of step (c). In one aspect, the amount of poly(vinyl alcohol) can be from about 0.5 wt. % to about 3.5 wt. % when a salt is present.

In another aspect, the amount of poly(vinyl alcohol) can be about 2 wt. % of the continuous phase when a salt is present.

In one embodiment of the process, the aqueous continuous phase of step (c) can comprise poly(vinyl alcohol) and salt and, optionally, the organic solvent used in the preparation of the dispersed phase solution in quantities up to saturating levels in the continuous phase solution.

The solvent phase of step (b) comprising poly(D,L-lactide) can comprise from about 10 wt. % to about 30 wt. % of polymer. In one aspect of this embodiment, the solvent phase can comprise from about 15 wt. % to about 25 wt. % of polymer. A non-limiting example of the disclosed process for forming microparticles of this aspect includes the following step (b):
b) providing an organic phase comprising about 20 wt. % poly(D,L-lactide).

One embodiment of the disclosed process for forming a microparticle of this aspect includes a process comprising:
a) providing an aqueous phase comprising from about 10 wt. % to about 30 wt. % ranibizumab and from about 0.1 wt. % to about 0.5 wt. % of poly(vinyl alcohol);
b) providing an organic phase comprising ethyl acetate and from about 10 wt. % to about 30 wt. % of poly(D,L-lactide);
c) combining the aqueous phase from step (a) with the organic phase from step (b) to form a primary emulsion;
d) combining the primary emulsion with an aqueous continuous phase comprising from about 1.5 wt. % to about 2.5 wt. % of poly(vinyl alcohol) to form a water/oil/water emulsion; and
e) combining the water/oil/water emulsion with an aqueous extraction phase and forming the microparticle.

Another embodiment of the disclosed process for forming a microparticle of this aspect includes a process comprising:
a) providing an aqueous phase comprising from about 10 wt. % to about 30 wt. % ranibizumab and from about 0.1 wt. % to about 0.5 wt. % of poly(vinyl alcohol);
b) providing an organic phase comprising ethyl acetate and from about 10 wt. % to about 30 wt. % of poly(D,L-lactide);
c) combining the aqueous phase from step (a) with the organic phase from step (b) to form a primary emulsion;
d) combining the primary emulsion with an aqueous continuous phase comprising ethyl acetate and from about 1.5 wt. % to about 2.5 wt. % of poly(vinyl alcohol) to form a water/oil/water emulsion; and
e) combining the water/oil/water emulsion with an aqueous extraction phase and forming the microparticle.

A further non-limiting example of the disclosed process for forming a microparticle of this aspect includes a process comprising:
a) providing an aqueous phase comprising from about 10 wt. % to about 30 wt. % ranibizumab and from about 0.1 wt. % to about 0.5 wt. % of poly(vinyl alcohol);
b) providing an organic phase comprising ethyl acetate and from about 10 wt. % to about 30 wt. % of poly(D,L-lactide) having an intrinsic viscosity of from about 0.3 dL/g to about 0.5 dL/g;
d) combining the aqueous phase from step (a) with the organic phase from step (b) to form a primary emulsion;
e) combining the primary emulsion with an aqueous continuous phase comprising from about 1.5 wt. % to about 2.5 wt. % of poly(vinyl alcohol) to form a water/oil/water emulsion; and
f) combining the water/oil/water emulsion with an aqueous extraction phase and forming the microparticle.

Yet another embodiment of the disclosed process for forming a microparticle of this aspect includes a process comprising:
a) providing an aqueous phase comprising from about 10 wt. % to about 30 wt. % ranibizumab and from about 0.1 wt. % to about 0.5 wt. % of poly(vinyl alcohol);
b) providing an organic phase comprising ethyl acetate and from about 10 wt. % to about 30 wt. % of poly(D,L-lactide) having an intrinsic viscosity of from about 0.3 dL/g to about 0.5 dL/g;
c) combining the aqueous phase from step (a) with the organic phase from step (b) to form a primary emulsion;
d) combining the primary emulsion with an aqueous continuous phase comprising methylene chloride and from about 1.5 wt. % to about 2.5 wt. % of poly(vinyl alcohol) to form a water/oil/water emulsion; and
e) combining the water/oil/water emulsion with an aqueous extraction phase and forming the microparticle.

A further non-limiting example of the disclosed process for forming a microparticle of this aspect includes a process comprising:
a) providing an aqueous phase comprising from about 10 wt. % to about 30 wt. % ranibizumab;
b) providing an organic phase comprising ethyl acetate and from about 10 wt. % to about 30 wt. % of poly(D,L-lactide) having an intrinsic viscosity of from about 0.3 dL/g to about 0.5 dL/g;
c) combining the aqueous phase from step (a) with the organic phase from step (b) to form a primary emulsion;
d) combining the primary emulsion with an aqueous continuous phase comprising from about 1.5 M to about 2.5 M NaCl to form a water/oil/water emulsion; and
e) combining the water/oil/water emulsion with an aqueous extraction phase and forming the microparticle.

A yet further non-limiting example of the disclosed process for forming a microparticle of this aspect includes a process comprising:
a) providing an aqueous phase comprising from about 10 wt. % to about 30 wt. % ranibizumab;
b) providing an organic phase comprising methylene chloride and from about 10 wt. % to about 30 wt. % of poly(D,L-lactide) having an intrinsic viscosity of from about 0.3 dL/g to about 0.5 dL/g;
c) combining the aqueous phase from step (a) with the organic phase from step (b) to form a primary emulsion;
d) combining the primary emulsion with an aqueous continuous phase comprising from about 1.5 M to about 2.5 M NaCl to form a water/oil/water emulsion; and
e) combining the water/oil/water emulsion with an aqueous extraction phase and forming the microparticle.

A still further non-limiting example of the disclosed process for forming a microparticle of this aspect includes a process comprising:
a) providing an aqueous phase comprising from about 10 wt. % to about 30 wt. % ranibizumab and from about 0.1 wt. % to about 0.5 wt. % of poly(vinyl alcohol);
b) providing an organic phase comprising ethyl acetate and from about 10 wt. % to about 30 wt. % of poly(D,L-lactide) having an intrinsic viscosity of from about 0.3 dL/g to about 0.5 dL/g;
c) combining the aqueous phase from step (a) with the organic phase from step (b) to form a primary emulsion;
d) combining the primary emulsion with an aqueous continuous phase comprising from about 1.5 M to about 2.5 M NaCl and from about 1.5 wt. % to about 2.5 wt. % poly(vinyl alcohol) to form a water/oil/water emulsion; and
e) combining the water/oil/water emulsion with an aqueous extraction phase and forming the microparticle.

A still yet further non-limiting example of the disclosed process for forming a microparticle of this aspect includes a process comprising:
a) providing an aqueous phase comprising from about 10 wt. % to about 30 wt. % ranibizumab and from about 0.1 wt. % to about 0.5 wt. % of poly(vinyl alcohol);
b) providing an organic phase comprising methylene chloride and from about 10 wt. % to about 30 wt. % of poly(D,L-lactide) having an intrinsic viscosity of from about 0.3 dL/g to about 0.5 dL/g;
c) combining the aqueous phase from step (a) with the organic phase from step (b) to form a primary emulsion;
d) combining the primary emulsion with an aqueous continuous phase comprising from about 1.5 M to about 2.5 M NaCl and from about 1.5 wt. % to about 2.5 wt. % poly(vinyl alcohol) to form a water/oil/water emulsion; and
e) combining the water/oil/water emulsion with an aqueous extraction phase and forming the microparticle.

Disclosed herein are methods for treating or preventing age related macular degeneration. Further disclosed are methods for treating macular degeneration and diseases, illnesses, or conditions relating to retinal edema and retinal neovascularization including increased or abnormal macular angiogenesis.

Another aspect relates to administering to a subject in need of treatment with a microparticle comprising:
a) from about 1 wt. % to about 15 wt. % ranibizumab; and
b) poly(D,L-lactide).

One embodiment of this aspect relates to a method for treating or preventing age related macular degeneration comprising, administering to a subject in need of treatment a composition comprising:

A) a microparticle comprising:
a) from about 1 wt. % to about 15 wt. % ranibizumab; and
b) poly(D,L-lactide); and

B) a pharmaceutically acceptable carrier.

A non-limiting example of this embodiment relates to a method for treating or preventing age related macular degeneration comprising, administering to a subject in need of treatment a composition comprising:

A) a microparticle comprising:
a) about 8 wt. % ranibizumab; and
b) poly(D,L-lactide-co-glycolide) having an intrinsic viscosity of from about 0.2 to about 0.8 dL/g; and

B) a pharmaceutically acceptable carrier.

A further aspect relates to administering to a subject in need of treatment with a pharmaceutical composition comprising:

A) from about 1 mg to about 500 mg of a microparticle comprising:
a) from about 1 wt. % to about 15 wt. % ranibizumab; and
b) poly(D,L-lactide-co-glycolide) having an intrinsic viscosity of from about 0.2 to about 0.8 dL/g; and

B) a pharmaceutically acceptable carrier;

wherein the composition contains from about 1 wt. % to about 50 wt. % of solids.

One embodiment of this aspect relates to a method for treating or preventing age related macular degeneration comprising, administering to a subject in need of treatment a pharmaceutical composition comprising:

A) from about 1 mg to about 500 mg of a microparticle comprising:
a) from about 1 wt. % to about 15 wt. % ranibizumab; and
b) poly(D,L-lactide-co-glycolide) having an intrinsic viscosity of from about 0.2 to about 0.8 dL/g; and

B) a pharmaceutically acceptable carrier;

wherein the composition contains from about 1 wt. % to about 50 wt. % of solids.

A third major aspect of the disclosed microparticles relate to microparticles that comprise:
a) from about 1 wt. % to about 15 wt. % ranibizumab; and
b) a polymer admixture comprising:
   i) from about 60% to about 99% poly(D,L-lactide); and
   ii) from about 1% to about 40% polycaprolactone.

The disclosed microparticles of this aspect comprise from about 1 wt. % to about 15 wt. % ranibizumab. In one embodiment of this aspect, the microparticles comprise from about 5 wt. % to about 10 wt. % of ranibizumab. In another embodiment, the microparticles comprise from about 2 wt. % to about 7 wt. % of ranibizumab. In a further embodiment, the microparticles comprise from about 4 wt. % to about 8 wt. % of ranibizumab. One example comprises from about 5 wt. % to about 6 wt. % of ranibizumab. The microparticles, however, can comprise any amount of ranibizumab from about 1 wt. % to about 15 wt. %, for example, 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, and 15 wt. %. Included in these amounts are fractional amounts of ranibizumab, for example, 1.1 wt. %, 2.75 wt. %, 4.33 wt. %, and the like.

The disclosed microparticles of this aspect comprise and admixture of poly(D,L-lactide) and polycaprolactone. The poly(D,L-lactide) can have an intrinsic viscosity (IV) of from about 0.3 to about 0.6 dL/g. In one embodiment, the copolymer has an IV of from about 0.3 to about 0.5 dL/g. In another embodiment, the copolymer has an IV of from about 0.4 to about 0.6 dL/g. In a further embodiment, the copolymer has an IV of from about 0.35 to about 0.55 dL/g. The microparticles, however, can comprise any IV from about 0.3 to about 0.6 dL/g, for example, 0.3 dL/g, 0.35 dL/g, 0.4 dL/g, 0.45 dL/g, 0.5 dL/g, 0.55 dL/g, 0.6 dL/g, and the like, or any fraction amount thereof, for example, 0.557 dL/g. Alternatively, the formulator can express the inherent viscosity in cm³/g if convenient.

The polycaprolactone can have an intrinsic viscosity (IV) of from 0.8 to about 1.2 dL/g. In one embodiment, the copolymer has an IV of from about 0.9 to about 1.2 dL/g. In a further embodiment, the copolymer has an IV of from about 0.8 to about 1.1 dL/g. In a yet further embodiment, the copolymer has an IV of from about 0.85 to about 1.0 dL/g. The microparticles, however, can comprise any IV from about 0.3 to about 0.6 dL/g, for example, 0.8 dL/g, 0.85 dL/g, 0.9 dL/g, 0.95 dL/g, 1.0 dL/g, 1.05 dL/g, 1.1 dL/g, 1.15 dL/g, 1.2 dL/g, and the like, or any fraction amount thereof, for example, 8.557 dL/g. Alternatively, the formulator can express the inherent viscosity in cm³/g if convenient.

The disclosed microparticles of this aspect have an average or mean particle size of from about 20 microns to about 125 microns. In one embodiment the range of mean particle size is from about 40 microns to about 90 microns. In another embodiment the range of mean particle sizes is from about 50 microns to about 80 microns. Particle size distributions are measured by laser diffraction techniques known to those of skill in the art.

In a further embodiment of this aspect, the bulk drug product that is used during preparation of the microparticles can comprise one or more water soluble carriers or excipients. Such carriers or excipients may generally include sugars, saccharides, polysaccharides, surfactants, buffer salts, bulking agents, and the like. A non-limiting example of an excipient is 2-(hydroxy-methyl)-6-[3,4,5-trihydroxy-6-(hydroxymethyl)tetrahydropyran-2-yl]oxy-tetrahydropyran-3,4,5-triol, “trehalose.” One embodiment of the disclosed process includes a bulk drug product used during preparation of the microparticles comprising 1 wt. % to 200 wt. % trehalose based on the weight of drug in the starting bulk drug product. In a further embodiment, the bulk drug product used during preparation of the microparticles comprises about 10 wt. % to 50 wt. % trehalose based on the weight of drug in the starting bulk drug product. In a non-limiting example of this aspect the bulk drug product comprises 25 wt % to 35 wt % trehalose. Another non-limiting example of an excipient is the surfactant polysorbate 20 (or Tween 20). One embodiment of the disclosed process includes a bulk drug product used during preparation of the microparticles comprising 0.01 wt % to 5 wt % polysorbate 20 based on the weight of drug in the starting bulk drug product. In a yet further embodiment, the bulk drug product used during preparation of the microparticles comprises about 0.05 wt % to 0.25 wt % polysorbate 20 based on the weight of drug in the starting bulk drug product. In a non-limiting example of this aspect the bulk drug product comprises about 0.1 wt % polysorbate 20. Further, the bulk drug product may contain two or more such carriers or excipients. A non-limiting example includes a bulk drug product comprising 25 wt % to 35 wt % trehalose and about 0.1 wt % polysorbate 20 based on the weight of drug in the starting bulk drug product

Another aspect of the disclosed compositions that can be used for treating age related macular degeneration relates to compositions comprising:

A) a microparticle comprising:
a) from about 1 wt. % to about 15 wt. % ranibizumab; and
b) a polymer blend comprising:
   i) from about 60 wt. % to about 99 wt. % poly(D,L-lactide); and
   ii) from about 1 wt. % to about 40 wt. % polycaprolactone; and

B) a pharmaceutically acceptable carrier.

In one embodiment, the composition comprises:

A) a microparticle comprising:
a) from about 5 wt. % to about 10 wt. % ranibizumab; and
b) a polymer blend comprising:
   i) from about 60 wt. % to about 99 wt. % poly(D,L-lactide) having an intrinsic viscosity of from about 0.3 to about 0.6 dL/g; and
   ii) from about 1 wt. % to about 40 wt. % polycaprolactone having an intrinsic viscosity of from about 0.8 to about 1.2 dL/g; and

B) a pharmaceutically acceptable carrier.

One example of this embodiment relates to a composition comprising:

A) a microparticle comprising:
a) from about 5 wt. % to about 10 wt. % ranibizumab; and
b) a polymer blend comprising:
   i) about 95 wt. % poly(D,L-lactide) having an intrinsic viscosity of from about 0.3 to about 0.6 dL/g; and
   ii) about 5 wt. % polycaprolactone having an intrinsic viscosity of from about 0.8 to about 1.2 dL/g; and

B) a pharmaceutically acceptable carrier.

In another example of this embodiment relates to a composition comprising:

A) a microparticle comprising:
a) from about 5 wt. % to about 10 wt. % ranibizumab; and
b) a polymer blend comprising:
   i) about 95 wt. % poly(D,L-lactide) having an intrinsic viscosity of 0.5 dL/g; and
   ii) about 5 wt. % polycaprolactone having an intrinsic viscosity of about 1.0 dL/g; and

B) a pharmaceutically acceptable carrier.

A further aspect relates to a pharmaceutical composition comprising:

A) from about 1 mg to about 500 mg of a microparticle comprising:
a) from about 1 wt. % to about 15 wt. % ranibizumab; and
b) a polymer blend comprising:
   i) from about 60 wt. % to about 99 wt. % poly(D,L-lactide) having an intrinsic viscosity of from about 0.3 to about 0.6 dL/g; and
   ii) from about 1 wt. % to about 40 wt. % polycaprolactone having an intrinsic viscosity of from about 0.8 to about 1.2 dL/g; and

B) a pharmaceutically acceptable carrier;

wherein the composition contains from about 1 wt. % to about 50 wt. % of solids.

The disclosed microparticles of this aspect can be prepared according to any microparticle preparation process. In one aspect, the microparticles are prepared according to U.S. Pat. No. 5,407,609 included herein by reference in its entirety. In another aspect, the process comprises:
a) providing an aqueous phase comprising from about 1 wt. % to about 50 wt. % ranibizumab;
b) providing an organic phase comprising a polymer admixture comprising:
   i) from about 60% to about 99% poly(D,L-lactide); and
   ii) from about 1% to about 40% polycaprolactone;
c) combining the aqueous phase from step (a) with the organic phase from step (b) to form a primary emulsion;
d) combining the primary emulsion with an aqueous continuous phase to form a water/oil/water emulsion; and
e) combining the water/oil/water emulsion with an aqueous extraction phase and forming the microparticle.

The disclosed process for forming the microparticles of this aspect can use in step (b) any non-halogenated hydrocarbon organic solvent or any halogenated organic solvent. In addition, a solvent can be added to the aqueous continuous phase of step (c) in an amount up to the point of saturation.

By “non-halogenated organic solvent” is meant an organic solvent suitable for serving as a primary solvent for dissolving the polymers disclosed herein, for example, in step (b). Non-limiting examples of non-halogenated solvents include: ketones, inter alia, acetone, methyl ethyl ketone; alcohols, inter alia, methanol, ethanol, n-propanol, iso-propanol, benzyl alcohol, glycerol; ethers, inter alia, diethyl ether, tetrahydrofuran, glyme, diglyme; esters, inter alia, methyl acetate, ethyl acetate; hydrocarbons, inter alia, n-pentane, iso-pentane, hexane, heptane, isooctane, benzene, toluene, xylene (all isomers); polar aprotic solvents, inter alia, dimethyl formamide, dimethyl acetamide, dimethyl sulfoxide, hexamethylphosphoramide, and the like.

By “halogenated solvents” is meant halogenated organic solvents, i.e., C₁-C₄ halogenated alkanes, non-limiting examples of which include carbon tetrachloride, chloroform, methylene chloride, chloroethane, 1,1-dichloroethane, 1,1,1-trichloroethane, and 1,2-dichloroethane.

In one aspect of the process for forming the disclosed microparticles of this aspect, ethyl acetate is used to dissolve the polymer admixture in step (b). In another aspect of the process for forming the disclosed microparticles of this aspect, methylene chloride is used to dissolve the polymer admixture in step (b).

In another embodiment of the process for forming the disclosed microparticles of this aspect, a salt can be added to the aqueous continuous phase of step (c) in an amount up to the point of saturation. A salt can be added alone or in combination with an organic solvent added to the continuous phase. In one aspect, a salt is present in an amount from about 0.1 M to about 10 M. In another aspect, a salt is present in an amount from about 0.5 M to about 5 M. However, the concentration of the salt in the continuous phase can be any amount from about 0.1 M to about 10 M. One example of a salt that can be used in the continuous phase is sodium chloride, for example, 1.5M NaCl, 2 M NaCl, 2.5 M NaCl, and the like. In one aspect of this embodiment, the aqueous continuous phase can comprise 2 M NaCl. A non-limiting example of the disclosed process for forming microparticles of this aspect includes the following step (d):
d) combining the primary emulsion (water in oil emulsion formed in step (c)) with an aqueous continuous phase comprising 2 M NaCl to form a water/oil/water emulsion.

In a yet further embodiment of the process for forming the disclosed microparticles of this aspect, a surfactant, for example, poly(vinyl alcohol) (PVA), can be added to the aqueous solution comprising ranibizumab of step (a), the continuous phase of step (c), or to both. The amount of PVA can be from about 0.05 wt. % to about 1 wt. % of the aqueous solution of step (a). The amount of surfactant can be from about 0.5 wt. % to about 3 wt. % of the continuous phase. In one aspect of this embodiment, the aqueous phase of step (a) comprises from about 0.1 wt. % to about 0.5 wt. % of poly(vinyl alcohol). In another aspect of this embodiment, the continuous phase comprises from about 1.5 wt. % to about 2.5 wt. % of poly(vinyl alcohol).

The aqueous Continuous Phase solution can comprise one or more other surfactants or emulsifiers. Other surfactants and emulsifying agents include most any physiologically acceptable emulsifiers. Examples include lecithin such as egg lecithin or soya bean lecithin or synthetic lecithins. Emulsifiers also include surfactants such as free fatty acids, esters of fatty acids of polyoxyalkylene compounds like polyoxpropylene glycol and polyoxyethylene glycol; ethers of fatty alcohols with polyoxyalkylene glycols; esters of fatty acids with polyoxyalkylated sorbitan; soaps; glycerol-polyalkylene stearate; glycerol-polyoxyethylene ricinoleate; homo- and co-polymers of polyalkylene glycols; polyethoxylated soya-oil and castor oil as well as hydrogenated derivatives; ethers and esters of sucrose or other carbohydrates with fatty acids, fatty alcohols, these being optionally polyoxyalkylated; mono-, di-, and tri-glycerides of saturated or unsaturated fatty acids, glycerides or soya-oil and sucrose. Other emulsifiers include natural and synthetic forms of bile salts or bile acids, both conjugated with amino acids and unconjugated such as taurodeoxycholate, and cholic acid

One embodiment of the process for preparing the microparticles of this aspect relates to the use of both a surfactant, for example, poly(vinyl alcohol) and a salt, for example, NaCl in the aqueous continuous phase of step (c). In one aspect, the amount of poly(vinyl alcohol) can be from about 0.5 wt. % to about 3.5 wt. % when a salt is present. In another aspect, the amount of poly(vinyl alcohol) can be about 2 wt. % of the continuous phase when a salt is present.

In one embodiment of the process, the aqueous continuous phase of step (c) can comprise poly(vinyl alcohol) and salt and, optionally, the organic solvent used in the preparation of the dispersed phase solution in quantities up to saturating levels in the continuous phase solution.

The solvent phase of step (b) comprising an admixture of poly(D,L-lactide) and polycaprolactone polymers can comprise from about 10 wt. % to about 30 wt. % of total polymer concentration. In one aspect of this embodiment, the solvent phase can comprise from about 15 wt. % to about 25 wt. % total polymer concentration. A non-limiting example of the disclosed process for forming microparticles of this aspect includes the following step (b):
b) providing an organic phase comprising about 20 wt. % of a polymer admixture comprising:
   i) from about 60% to about 99% poly(D,L-lactide); and
   ii) from about 1% to about 40% polycaprolactone.

One embodiment of the disclosed process for forming a microparticle of this aspect includes a process comprising:
a) providing an aqueous phase comprising from about 10 wt. % to about 30 wt. % ranibizumab and from about 0.1 wt. % to about 0.5 wt. % of poly(vinyl alcohol);
b) providing an organic phase comprising ethyl acetate and about 20 wt. % of a polymer admixture comprising:
   i) from about 60% to about 99% poly(D,L-lactide) having an intrinsic viscosity of from about 0.3 dL/g to about 0.6 dL/g; and
   ii) from about 1% to about 40% polycaprolactone having an intrinsic viscosity of from about 0.8 dL/g to about 1.2 dL/g;
c) combining the aqueous phase from step (a) with the organic phase from step (b) to form a primary emulsion;
d) combining the primary emulsion with an aqueous continuous phase comprising from about 1.5 wt. % to about 2.5 wt. % of poly(vinyl alcohol) to form a water/oil/water emulsion; and
e) combining the water/oil/water emulsion with an aqueous extraction phase and forming the microparticle.

Another embodiment of the disclosed process for forming a microparticle of this aspect includes a process comprising:
a) providing an aqueous phase comprising from about 10 wt. % to about 30 wt. % ranibizumab and from about 0.1 wt. % to about 0.5 wt. % of poly(vinyl alcohol);
b) providing an organic phase comprising about 20 wt. % of a polymer admixture comprising:
   i) from about 60% to about 99% poly(D,L-lactide); and
   ii) from about 1% to about 40% polycaprolactone;
c) combining the aqueous phase from step (a) with the organic phase from step (b) to form a primary emulsion;
d) combining the primary emulsion with an aqueous continuous phase comprising ethyl acetate and from about 1.5 wt. % to about 2.5 wt. % of poly(vinyl alcohol) to form a water/oil/water emulsion; and
e) combining the water/oil/water emulsion with an aqueous extraction phase and forming the microparticle.

A further non-limiting example of the disclosed process for forming a microparticle of this aspect includes a process comprising:
a) providing an aqueous phase comprising from about 10 wt. % to about 30 wt. % ranibizumab and from about 0.1 wt. % to about 0.5 wt. % of poly(vinyl alcohol);
b) providing an organic phase comprising methylene chloride and about 20 wt. % of a polymer admixture comprising:
   i) from about 60% to about 99% poly(D,L-lactide) having an intrinsic viscosity of from about 0.3 dL/g to about 0.6 dL/g; and
   ii) from about 1% to about 40% polycaprolactone having an intrinsic viscosity of from about 0.8 dL/g to about 1.2 dL/g;
c) combining the aqueous phase from step (a) with the organic phase from step (b) to form a primary emulsion;
d) combining the primary emulsion with an aqueous continuous phase comprising from about 1.5 wt. % to about 2.5 wt. % of poly(vinyl alcohol) to form a water/oil/water emulsion; and
e) combining the water/oil/water emulsion with an aqueous extraction phase and forming the microparticle.

Yet another embodiment of the disclosed process for forming a microparticle of this aspect includes a process comprising:
a) providing an aqueous phase comprising from about 10 wt. % to about 30 wt. % ranibizumab and from about 0.1 wt. % to about 0.5 wt. % of poly(vinyl alcohol);
b) providing an organic phase comprising ethyl acetate and about 20 wt. % of a polymer admixture comprising:
   i) from about 60% to about 99% poly(D,L-lactide) having an intrinsic viscosity of from about 0.3 dL/g to about 0.6 dL/g; and
   ii) from about 1% to about 40% polycaprolactone having an intrinsic viscosity of from about 0.8 dL/g to about 1.2 dL/g;
c) combining the aqueous phase from step (a) with the organic phase from step (b) to form a primary emulsion;
d) combining the primary emulsion with an aqueous continuous phase comprising methylene chloride and from about 1.5 wt. % to about 2.5 wt. % of poly(vinyl alcohol) to form a water/oil/water emulsion; and
e) combining the water/oil/water emulsion with an aqueous extraction phase and forming the microparticle.

A further non-limiting example of the disclosed process for forming a microparticle of this aspect includes a process comprising:
a) providing an aqueous phase comprising from about 10 wt. % to about 30 wt. % ranibizumab;
b) providing an organic phase comprising ethyl acetate and about 20 wt. % of a polymer admixture comprising:
   i) from about 60% to about 99% poly(D,L-lactide) having an intrinsic viscosity of from about 0.3 dL/g to about 0.6 dL/g; and
   ii) from about 1% to about 40% polycaprolactone having an intrinsic viscosity of from about 0.8 dL/g to about 1.2 dL/g;
c) combining the aqueous phase from step (a) with the organic phase from step (b) to form a primary emulsion;
d) combining the primary emulsion with an aqueous continuous phase comprising from about 1.5 M to about 2.5 M NaCl to form a water/oil/water emulsion; and
e) combining the water/oil/water emulsion with an aqueous extraction phase and forming the microparticle.

A yet further non-limiting example of the disclosed process for forming a microparticle of this aspect includes a process comprising:
a) providing an aqueous phase comprising from about 10 wt. % to about 30 wt. % ranibizumab;
b) providing an organic phase comprising methylene chloride and about 20 wt. % of a polymer admixture comprising:
   i) from about 60% to about 99% poly(D,L-lactide) having an intrinsic viscosity of from about 0.3 dL/g to about 0.6 dL/g; and
   ii) from about 1% to about 40% polycaprolactone having an intrinsic viscosity of from about 0.8 dL/g to about 1.2 dL/g;
c) combining the aqueous phase from step (a) with the organic phase from step (b) to form a primary emulsion;
d) combining the primary emulsion with an aqueous continuous phase comprising from about 1.5 M to about 2.5 M NaCl to form a water/oil/water emulsion; and
e) combining the water/oil/water emulsion with an aqueous extraction phase and forming the microparticle.

A still further non-limiting example of the disclosed process for forming a microparticle of this aspect includes a process comprising:
a) providing an aqueous phase comprising from about 10 wt. % to about 30 wt. % ranibizumab and from about 0.1 wt. % to about 0.5 wt. % of poly(vinyl alcohol);
b) providing an organic phase comprising ethyl acetate and about 20 wt. % of a polymer admixture comprising:
   i) from about 60% to about 99% poly(D,L-lactide) having an intrinsic viscosity of from about 0.3 dL/g to about 0.6 dL/g; and
   ii) from about 1% to about 40% polycaprolactone having an intrinsic viscosity of from about 0.8 dL/g to about 1.2 dL/g;
c) combining the aqueous phase from step (a) with the organic phase from step (b) to form a primary emulsion;
d) combining the primary emulsion with an aqueous continuous phase comprising from about 1.5 M to about 2.5 M NaCl and from about 1.5 wt. % to about 2.5 wt. % poly(vinyl alcohol) to form a water/oil/water emulsion; and
e) combining the water/oil/water emulsion with an aqueous extraction phase and forming the microparticle.

A still yet further non-limiting example of the disclosed process for forming a microparticle of this aspect includes a process comprising:
a) providing an aqueous phase comprising from about 10 wt. % to about 30 wt. % ranibizumab and from about 0.1 wt. % to about 0.5 wt. % of poly(vinyl alcohol);
b) providing an organic phase comprising ethyl acetate and about 20 wt. % of a polymer admixture comprising:
   i) from about 60% to about 99% poly(D,L-lactide) having an intrinsic viscosity of from about 0.3 dL/g to about 0.6 dL/g; and
   ii) from about 1% to about 40% polycaprolactone having an intrinsic viscosity of from about 0.8 dL/g to about 1.2 dL/g;
c) combining the aqueous phase from step (a) with the organic phase from step (b) to form a primary emulsion;
d) combining the primary emulsion with an aqueous continuous phase comprising from about 1.5 M to about 2.5 M NaCl and from about 1.5 wt. % to about 2.5 wt. % poly(vinyl alcohol) to form a water/oil/water emulsion; and
e) combining the water/oil/water emulsion with an aqueous extraction phase and forming the microparticle.

Disclosed herein are methods for treating or preventing age related macular degeneration. Further disclosed are methods for treating macular degeneration and diseases, illnesses, or conditions relating to retinal edema and retinal neovascularization including increased or abnormal macular angiogenesis.

A further aspect relates to a method for treating or preventing macular degeneration comprising administering to a subject a composition comprising:

A) a microparticle comprising:
a) from about 1 wt. % to about 15 wt. % ranibizumab; and
b) a polymer blend comprising:
   i) from about 60 wt. % to about 99 wt. % poly(D,L-lactide); and
   ii) from about 1 wt. % to about 40 wt. % polycaprolactone; and

B) a pharmaceutically acceptable carrier.

In one embodiment, the disclosed method relates to administering a composition comprising:

A) a microparticle comprising:
a) from about 5 wt. % to about 10 wt. % ranibizumab; and
b) a polymer blend comprising:
   i) from about 60 wt. % to about 99 wt. % poly(D,L-lactide); and
   ii) from about 1 wt. % to about 40 wt. % polycaprolactone; and

B) a pharmaceutically acceptable carrier.

One example of this embodiment relates to administering compositions comprising:

A) a microparticle comprising:
a) from about 5 wt. % to about 10 wt. % ranibizumab; and
b) a polymer blend comprising:
   i) from about 60 wt. % to about 99 wt. % poly(D,L-lactide) having an intrinsic viscosity of from about 0.3 to about 0.6 dL/g; and
   ii) from about 1 wt. % to about 40 wt. % polycaprolactone having an intrinsic viscosity of from about 0.8 to about 1.2 dL/g; and

B) a pharmaceutically acceptable carrier.

In another embodiment, the composition comprises:

A) a microparticle comprising:
a) from about 5 wt. % to about 10 wt. % ranibizumab; and
b) a polymer blend comprising:
   i) about 95 wt. % poly(D,L-lactide) having an intrinsic viscosity of from about 0.3 to about 0.6 dL/g; and
   ii) about 5 wt. % polycaprolactone having an intrinsic viscosity of from about 0.8 to about 1.2 dL/g; and

B) a pharmaceutically acceptable carrier.

A further embodiment relates to a method for treating or preventing macular degeneration comprising administering to a subject a composition comprising:

A) from about 1 mg to about 500 mg of a microparticle comprising:
a) from about 1 wt. % to about 15 wt. % ranibizumab; and
b) a polymer blend comprising:
   i) from about 60 wt. % to about 99 wt. % poly(D,L-lactide); and
   ii) from about 1 wt. % to about 40 wt. % polycaprolactone; and

B) a pharmaceutically acceptable carrier;

wherein the composition contains from about 1 wt. % to about 50 wt. % of solids.

A still further embodiment relates to a method for treating or preventing macular degeneration comprising administering to a subject a composition comprising:

A) from about 1 mg to about 500 mg of a microparticle comprising:
a) from about 1 wt. % to about 15 wt. % ranibizumab; and
b) a polymer blend comprising:
   i) from about 60 wt. % to about 99 wt. % poly(D,L-lactide); and
   ii) from about 1 wt. % to about 40 wt. % polycaprolactone; and

B) a pharmaceutically acceptable carrier;

wherein the composition contains from about 1 wt. % to about 50 wt. % of solids.

A fourth major aspect of the disclosed microparticles relate to microparticles that comprise:
a) from about 1 wt. % to about 15 wt. % ranibizumab; and
b) an admixture of poly(D,L-lactide-co-glycolide) copolymers wherein the admixture comprises:
   i) from about 10 wt. % to about 90 wt. % of a first copolymer wherein the first copolymer comprises from about 65% to about 90% D,L-lactide units and from 10% to about 35% glycolide units; and
   ii) from about 10 wt. % to about 90 wt. % of a second copolymer wherein the second copolymer comprises from about 55% to about 90% D,L-lactide units and from 10% to about 45% glycolide units.

The disclosed microparticles of this aspect comprise from about 1 wt. % to about 15 wt. % ranibizumab. In one embodiment of this aspect, the microparticles comprise from about 7 wt. % to about 10 wt. % of ranibizumab. In another embodiment, the microparticles comprise from about 2 wt. % to about 7 wt. % of ranibizumab. In a further embodiment, the microparticles comprise from about 4 wt. % to about 8 wt. % of ranibizumab. One example comprises from about 5 wt. % to about 6 wt. % of ranibizumab. The microparticles, however, can comprise any amount of ranibizumab from about 1 wt. % to about 15 wt. %, for example, 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, and 15 wt. %. Included in these amounts are fractional amounts of ranibizumab, for example, 1.1 wt. %, 2.75 wt. %, 4.33 wt. %, and the like.

The disclosed microparticles of this aspect comprise an admixture of a first poly(D,L-lactide-co-glycolides) and a second poly(D,L-lactide-co-glycolide). Each of the poly(D,L-lactide-co-glycolide) copolymers can have an intrinsic viscosity (IV) of from about 0.1 to about 0.9 dL/g. In one embodiment, one copolymer has an IV of from about 0.15 to about 0.25 dL/g and the other copolymer has an IV of from about 0.45 to about 0.85 dL/g. In another embodiment, one copolymer has an IV of from about 0.35 to about 0.65 dL/g and the other copolymer has an IV of from about 0.5 to about 0.7 dL/g. In a further embodiment, one copolymer has an IV of from about 0.1 to about 0.4 dL/g and the other copolymer has an IV of from about 0.5 to about 0.9 dL/g. The microparticles, however, can comprise any IV from about 0.3 to about 0.6 dL/g, for example, 0.3 dL/g, 0.35 dL/g, 0.4 dL/g, 0.45 dL/g, 0.5 dL/g, 0.55 dL/g, 0.6 dL/g, and the like, or any fraction amount thereof, for example, 0.557 dL/g. Alternatively, the formulator can express the inherent viscosity in cm³/g if convenient.

The disclosed microparticles of this aspect have an average or mean particle size of from about 20 microns to about 125 microns. In one embodiment the range of mean particle size is from about 40 microns to about 90 microns. In antother embodiment the range of mean particle sizes is from about 50 microns to about 80 microns. Particle size distributions are measured by laser diffraction techniques known to those of skill in the art.

In a further embodiment of this aspect, the bulk drug product that is used during preparation of the microparticles can comprise one or more water soluble carriers or excipients. Such carriers or excipients may generally include sugars, saccharides, polysaccharides, surfactants, buffer salts, bulking agents, and the like. A non-limiting example of an excipient is 2-(hydroxy-methyl)-6-[3,4,5-trihydroxy-6-(hydroxymethyl)tetrahydropyran-2-yl]oxy-tetrahydropyran-3,4,5-triol, “trehalose.” One embodiment of the disclosed process includes a bulk drug product used during preparation of the microparticles comprising 1 wt. % to 200 wt. % trehalose based on the weight of drug in the starting bulk drug product. In a further embodiment, the bulk drug product used during preparation of the microparticles comprises about 10 wt % to 50 wt % trehalose based on the weight of drug in the starting bulk drug product. In a non-limiting example of this aspect the bulk drug product comprises 25 wt % to 35 wt % trehalose. Another non-limiting example of an excipient is the surfactant polysorbate 20 (or Tween 20). One embodiment of the disclosed process includes a bulk drug product used during preparation of the microparticles comprising 0.01 wt % to 5 wt % polysorbate 20 based on the weight of drug in the starting bulk drug product. In a yet further embodiment, the bulk drug product used during preparation of the microparticles comprises about 0.05 wt % to 0.25 wt % polysorbate 20 based on the weight of drug in the starting bulk drug product. In a non-limiting example of this aspect the bulk drug product comprises about 0.1 wt % polysorbate 20. Further, the bulk drug product may contain two or more such carriers or excipients. A non-limiting example includes a bulk drug product comprising 25 wt % to 35 wt % trehalose and about 0.1 wt % polysorbate 20 based on the weight of drug in the starting bulk drug product

Another aspect of the disclosed compositions that can be used for treating age related macular degeneration relates to compositions comprising:

A) a microparticle comprising:
a) from about 1 wt. % to about 15 wt. % ranibizumab; and
b) a poly(D,L-lactide-co-glycolide) copolymer admixture comprising:
   i) from about 10 wt. % to about 90 wt. % of a first copolymer wherein the first copolymer comprises from about 65% to about 90% D,L-lactide units and from 10% to about 35% glycolide units; and
   ii) from about 10 wt. % to about 90 wt. % of a second copolymer wherein the second copolymer comprises from about 55% to about 90% D,L-lactide units and from 10% to about 45% glycolide units; and

B) a pharmaceutically acceptable carrier.

In one embodiment, the composition comprises:

A) a microparticle comprising:
a) from about 5 wt. % to about 10 wt. % ranibizumab; and
b) a poly(D,L-lactide-co-glycolide) copolymer admixture comprising:
   i) from about 10 wt. % to about 90 wt. % of a first copolymer wherein the first copolymer comprises from about 65% to about 90% D,L-lactide units and from 10% to about 35% glycolide units having an intrinsic viscosity of from about 0.4 dL/g to about 0.8 dL/g; and
   ii) from about 10 wt. % to about 90 wt. % of a second copolymer wherein the second copolymer comprises from about 55% to about 90% D,L-lactide units and from 10% to about 45% glycolide units having an intrinsic viscosity of from about 0.2 dL/g to about 0.7 dL/g; and

B) a pharmaceutically acceptable carrier.

In this, and other embodiments, one copolymer can be an ester capped poly(D,L-lactide-co-glycolide) and the other copolymer can be an acid capped poly(D,L-lactide-co-glycolide). Alternatively, both poly(D,L-lactide-co-glycolide) copolymers can be ester capped or both poly(D,L-lactide-co-glycolide) copolymers can be acid capped.

One example of this embodiment relates to a composition comprising:

A) a microparticle comprising:
a) from about 5 wt. % to about 10 wt. % ranibizumab; and
b) a poly(D,L-lactide-co-glycolide) copolymer admixture comprising:
   i) about 50 wt. % of an ester capped poly(D,L-lactide-co-glycolide) comprising 85% D,L-lactide units and 15% glycolide units, wherein the copolymer has an intrinsic viscosity of from about 0.4 dL/g to about 0.5 dL/g; and
   ii) about 50 wt. % of an acid capped poly(D,L-lactide-co-glycolide) comprising 85% D,L-lactide units and 15% glycolide units, wherein the copolymer has an intrinsic viscosity of from about 0.5 dL/g to about 0.7 dL/g; and

B) a pharmaceutically acceptable carrier.

In another example of this embodiment relates to a composition comprising:

A) a microparticle comprising:
a) from about 5 wt. % to about 10 wt. % ranibizumab; and
b) a poly(D,L-lactide-co-glycolide) copolymer admixture comprising:
   i) about 80 wt. % of an ester capped poly(D,L-lactide-co-glycolide) comprising 75% D,L-lactide units and 25% glycolide units, wherein the copolymer has an intrinsic viscosity of from about 0.65 dL/g to about 0.75 dL/g; and
   ii) about 20 wt. % of an acid capped poly(D,L-lactide-co-glycolide) comprising 65% D,L-lactide units and 35% glycolide units, wherein the copolymer has an intrinsic viscosity of from about 0.35 dL/g to about 0.55 dL/g; and

B) a pharmaceutically acceptable carrier.

In another example of this embodiment relates to a composition comprising:

A) a microparticle comprising:
a) from about 5 wt. % to about 10 wt. % ranibizumab; and
b) a poly(D,L-lactide-co-glycolide) copolymer admixture comprising:
   i) about 80 wt. % of an acid capped poly(D,L-lactide-co-glycolide) comprising 75% D,L-lactide units and 25% glycolide units, wherein the copolymer has an intrinsic viscosity of from about 0.65 dL/g to about 0.75 dL/g; and
   ii) about 20 wt. % of an ester capped poly(D,L-lactide-co-glycolide) comprising 65% D,L-lactide units and 35% glycolide units, wherein the copolymer has an intrinsic viscosity of from about 0.15 dL/g to about 0.25 dL/g; and

B) a pharmaceutically acceptable carrier.

A further aspect relates to a pharmaceutical composition comprising:

A) from about 1 mg to about 500 mg of a microparticle comprising:
a) from about 1 wt. % to about 15 wt. % ranibizumab; and
b) a poly(D,L-lactide-co-glycolide) copolymer admixture comprising:
   i) about 50 wt. % of an acid capped poly(D,L-lactide-co-glycolide) comprising 85% D,L-lactide units and 15% glycolide units, wherein the copolymer has an intrinsic viscosity of from about 0.45 dL/g to about 0.55 dL/g; and
   ii) about 50 wt. % of an ester capped poly(D,L-lactide-co-glycolide) comprising 75% D,L-lactide units and 25% glycolide units, wherein the copolymer has an intrinsic viscosity of from about 0.45 dL/g to about 0.60 dL/g, or from about 0.45 dL/g to about 5.5 dL/g, or from about 0.50 dL/g to about 0.60 dL/g; and

B) a pharmaceutically acceptable carrier;

wherein the composition contains from about 1 wt. % to about 50 wt. % of solids.

The disclosed microparticles of this aspect can be prepared according to any microparticle preparation process. In one aspect, the microparticles are prepared according to U.S. Pat. No. 5,407,609 included herein by reference in its entirety. In another aspect, the process comprises:
a) providing an aqueous phase comprising from about 1 wt. % to about 50 wt. % ranibizumab;
b) providing an organic phase comprising an admixture of poly(D,L-lactide-co-glycolide) copolymers wherein the admixture comprises:
   i) from about 10% to about 90% of a first copolymer wherein the first copolymer comprises from about 65% to about 90% D,L-lactide units and from 10% to about 35% glycolide units; and ii) from about 10% to about 90% of a second copolymer wherein the second copolymer comprises from about 55% to about 90% D,L-lactide units and from 10% to about 45% glycolide units;
c) combining the aqueous phase from step (a) with the organic phase from step (b) to form a primary emulsion;
d) combining the primary emulsion with an aqueous continuous phase to form a water/oil/water emulsion; and
e) combining the water/oil/water emulsion with an aqueous extraction phase and forming the microparticle.

The disclosed process for forming the microparticles of this aspect can use in step (b) any non-halogenated hydrocarbon organic solvent or any halogenated organic solvent. In addition, a solvent can be added to the aqueous continuous phase of step (c) in an amount up to the point of saturation.

By “non-halogenated organic solvent” is meant an organic solvent suitable for serving as a primary solvent for dissolving the polymers disclosed herein, for example, in step (b). Non-limiting examples of non-halogenated solvents include: ketones, inter alia, acetone, methyl ethyl ketone; alcohols, inter alia, methanol, ethanol, n-propanol, iso-propanol, benzyl alcohol, glycerol; ethers, inter alia, diethyl ether, tetrahydrofuran, glyme, diglyme; esters, inter alia, methyl acetate, ethyl acetate; hydrocarbons, inter alia, n-pentane, iso-pentane, hexane, heptane, isooctane, benzene, toluene, xylene (all isomers); polar aprotic solvents, inter alia, dimethyl formamide, dimethyl acetamide, dimethyl sulfoxide, hexamethylphosphoramide, and the like.

By “halogenated solvents” is meant halogenated organic solvents, i.e., C₁-C₄ halogenated alkanes, non-limiting examples of which include carbon tetrachloride, chloroform, methylene chloride, chloroethane, 1,1-dichloroethane, 1,1,1-trichloroethane, and 1,2-dichloroethane.

In one aspect of the process for forming the disclosed microparticles of this aspect, ethyl acetate is used to dissolve the polymer admixture in step (b). In another aspect of the process for forming the disclosed microparticles of this aspect, methylene chloride is used to dissolve the polymer admixture in step (b).

In another embodiment of the process for forming the disclosed microparticles of this aspect, a salt can be added to the aqueous continuous phase of step (c) in an amount up to the point of saturation. A salt can be added alone or in combination with an organic solvent added to the continuous phase. In one aspect, a salt is present in an amount from about 0.1 M to about 10 M. In another aspect, a salt is present in an amount from about 0.5 M to about 5 M. However, the concentration of the salt in the continuous phase can be any amount from about 0.1 M to about 10 M. One example of a salt that can be used in the continuous phase is sodium chloride, for example, 1.5M NaCl, 2 M NaCl, 2.5 M NaCl, and the like. In one aspect of this embodiment, the aqueous continuous phase can comprise 2 M NaCl. A non-limiting example of the disclosed process for forming microparticles of this aspect includes the following step (d):
d) combining the primary emulsion (water in oil emulsion formed in step (c)) with an aqueous continuous phase comprising 2 M NaCl to form a water/oil/water emulsion.

In a yet further embodiment of the process for forming the disclosed microparticles of this aspect, a surfactant, for example, poly(vinyl alcohol) (PVA), can be added to the aqueous solution comprising ranibizumab of step (a), the continuous phase of step (c), or to both. The amount of PVA can be from about 0.05 wt. % to about 1 wt. % of the aqueous solution of step (a). The amount of surfactant can be from about 0.5 wt. % to about 3 wt. % of the continuous phase. In one aspect of this embodiment, the aqueous phase of step (a) comprises from about 0.1 wt. % to about 0.5 wt. % of poly(vinyl alcohol). In another aspect of this embodiment, the continuous phase comprises from about 1.5 wt. % to about 2.5 wt. % of poly(vinyl alcohol).

The aqueous Continuous Phase solution can comprise one or more other surfactants or emulsifiers. Other surfactants and emulsifying agents include most any physiologically acceptable emulsifiers. Examples include lecithin such as egg lecithin or soya bean lecithin or synthetic lecithins. Emulsifiers also include surfactants such as free fatty acids, esters of fatty acids of polyoxyalkylene compounds like polyoxpropylene glycol and polyoxyethylene glycol; ethers of fatty alcohols with polyoxyalkylene glycols; esters of fatty acids with polyoxyalkylated sorbitan; soaps; glycerol-polyalkylene stearate; glycerol-polyoxyethylene ricinoleate; homo- and co-polymers of polyalkylene glycols; polyethoxylated soya-oil and castor oil as well as hydrogenated derivatives; ethers and esters of sucrose or other carbohydrates with fatty acids, fatty alcohols, these being optionally polyoxyalkylated; mono-, di-, and tri-glycerides of saturated or unsaturated fatty acids, glycerides or soya-oil and sucrose. Other emulsifiers include natural and synthetic forms of bile salts or bile acids, both conjugated with amino acids and unconjugated such as taurodeoxycholate, and cholic acid

One embodiment of the process for preparing the microparticles of this aspect relates to the use of both a surfactant, for example, poly(vinyl alcohol) and a salt, for example, NaCl in the aqueous continuous phase of step (c). In one aspect, the amount of poly(vinyl alcohol) can be from about 0.5 wt. % to about 3.5 wt. % when a salt is present. In another aspect, the amount of poly(vinyl alcohol) can be about 2 wt. % of the continuous phase when a salt is present.

In one embodiment of the process, the aqueous continuous phase of step (c) can comprise poly(vinyl alcohol) and salt and, optionally, the organic solvent used in the preparation of the dispersed phase solution in quantities up to saturating levels in the continuous phase solution.

Another embodiment relates to a microparticle comprising:
a) A first copolymer comprising:
   i) from about 65% to about 90% D,L-lactide units; and
   ii) from about 10% to about 35% glycolide units; and
b) a second copolymer comprising:
   i) from about 55% to about 90% D,L-lactide units; and
   ii) from about 10% to about 45% glycolide units.

The solvent phase of step (b) comprising an admixture of two or more poly(D,L-lactide-co-glycolide) copolymers can comprise from about 10 wt. % to about 30 wt. % of total polymer concentration. In one aspect of this embodiment, the solvent phase can comprise from about 15 wt. % to about 25 wt. % of total polymer concentration. A non-limiting example of the disclosed process for forming microparticles of this aspect includes the following step (b):
b) providing an organic phase comprising about 20 wt. % of a polymer admixture comprising:
   i) from about 10% to about 90% of a first copolymer wherein the first copolymer comprises from about 65% to about 90% D,L-lactide units and from 10% to about 35% glycolide units; and
   ii) from about 10% to about 90% of a second copolymer wherein the second copolymer comprises from about 55% to about 90% D,L-lactide units and from 10% to about 45% glycolide units.

One embodiment of the disclosed process for forming a microparticle of this aspect includes a process comprising:
a) providing an aqueous phase comprising from about 10 wt. % to about 30 wt. % ranibizumab and from about 0.1 wt. % to about 0.5 wt. % of poly(vinyl alcohol);
b) providing an organic phase comprising ethyl acetate and about 20 wt. % of a poly(D,L-lactide-co-glycolide) copolymer admixture comprising:
   i) from about 10% to about 90% of a first copolymer wherein the first copolymer comprises from about 65% to about 90% D,L-lactide units and from 10% to about 35% glycolide units; and
   ii) from about 10% to about 90% of a second copolymer wherein the second copolymer comprises from about 55% to about 90% D,L-lactide units and from 10% to about 45% glycolide units;
c) combining the aqueous phase from step (a) with the organic phase from step (b) to form a primary emulsion;
d) combining the primary emulsion with an aqueous continuous phase comprising from about 1.5 wt. % to about 2.5 wt. % of poly(vinyl alcohol) to form a water/oil/water emulsion; and
e) combining the water/oil/water emulsion with an aqueous extraction phase and forming the microparticle.

Another embodiment of the disclosed process for forming a microparticle of this aspect includes a process comprising:
a) providing an aqueous phase comprising from about 10 wt. % to about 30 wt. % ranibizumab and from about 0.1 wt. % to about 0.5 wt. % of poly(vinyl alcohol);
b) providing an organic phase comprising about 20 wt. % of a poly(D,L-lactide-co-glycolide) copolymer admixture comprising:
   i) from about 10% to about 90% of a first copolymer wherein the first copolymer comprises from about 65% to about 90% D,L-lactide units and from 10% to about 35% glycolide units; and
   ii) from about 10% to about 90% of a second copolymer wherein the second copolymer comprises from about 50% to about 75% D,L-lactide units and from 25% to about 50% glycolide units;
c) combining the aqueous phase from step (a) with the organic phase from step (b) to form a primary emulsion;
d) combining the primary emulsion with an aqueous continuous phase comprising ethyl acetate and from about 1.5 wt. % to about 2.5 wt. % of poly(vinyl alcohol) to form a water/oil/water emulsion; and
e) combining the water/oil/water emulsion with an aqueous extraction phase and forming the microparticle.

A further non-limiting example of the disclosed process for forming a microparticle of this aspect includes a process comprising:
a) providing an aqueous phase comprising from about 10 wt. % to about 30 wt. % ranibizumab and from about 0.1 wt. % to about 0.5 wt. % of poly(vinyl alcohol);
b) providing an organic phase comprising about 20 wt. % of a poly(D,L-lactide-co-glycolide) copolymer admixture comprising:
   i) from about 10% to about 90% of a first copolymer wherein the first copolymer comprises from about 65% to about 90% D,L-lactide units and from 10% to about 35% glycolide units; and
   ii) from about 10% to about 90% of a second copolymer wherein the second copolymer comprises from about 55% to about 90% D,L-lactide units and from 10% to about 45% glycolide units;
c) combining the aqueous phase from step (a) with the organic phase from step (b) to form a primary emulsion;
d) combining the primary emulsion with an aqueous continuous phase comprising from about 1.5 wt. % to about 2.5 wt. % of poly(vinyl alcohol) to form a water/oil/water emulsion; and
e) combining the water/oil/water emulsion with an aqueous extraction phase and forming the microparticle.

Yet another embodiment of the disclosed process for forming a microparticle of this aspect includes a process comprising:
a) providing an aqueous phase comprising from about 10 wt. % to about 30 wt. % ranibizumab and from about 0.1 wt. % to about 0.5 wt. % of poly(vinyl alcohol);
b) providing an organic phase comprising about 20 wt. % of a poly(D,L-lactide-co-glycolide) copolymer admixture comprising:
   i) from about 10% to about 90% of a first copolymer wherein the first copolymer comprises from about 65% to about 90% D,L-lactide units and from 10% to about 35% glycolide units; and
   ii) from about 10% to about 90% of a second copolymer wherein the second copolymer comprises from about 55% to about 90% D,L-lactide units and from 10% to about 45% glycolide units;
c) combining the aqueous phase from step (a) with the organic phase from step (b) to form a primary emulsion;
d) combining the primary emulsion with an aqueous continuous phase comprising methylene chloride and from about 1.5 wt. % to about 2.5 wt. % of poly(vinyl alcohol) to form a water/oil/water emulsion; and
e) combining the water/oil/water emulsion with an aqueous extraction phase and forming the microparticle.

A further non-limiting example of the disclosed process for forming a microparticle of this aspect includes a process comprising:
a) providing an aqueous phase comprising from about 10 wt. % to about 30 wt. % ranibizumab;
b) providing an organic phase comprising about 20 wt. % of a poly(D,L-lactide-co-glycolide) copolymer admixture comprising:
   i) from about 10% to about 90% of a first copolymer wherein the first copolymer comprises from about 65% to about 90% D,L-lactide units and from 10% to about 35% glycolide units; and
   ii) from about 10% to about 90% of a second copolymer wherein the second copolymer comprises from about 55% to about 90% D,L-lactide units and from 10% to about 45% glycolide units;
c) combining the aqueous phase from step (a) with the organic phase from step (b) to form a primary emulsion;
d) combining the primary emulsion with an aqueous continuous phase comprising from about 1.5 M to about 2.5 M NaCl to form a water/oil/water emulsion; and
e) combining the water/oil/water emulsion with an aqueous extraction phase and forming the microparticle.

A yet further non-limiting example of the disclosed process for forming a microparticle of this aspect includes a process comprising:
a) providing an aqueous phase comprising from about 10 wt. % to about 30 wt. % ranibizumab;
b) providing an organic phase comprising about 20 wt. % of a poly(D,L-lactide-co-glycolide) copolymer admixture comprising:
   i) from about 10% to about 90% of a first copolymer wherein the first copolymer comprises from about 65% to about 90% D,L-lactide units and from 10% to about 35% glycolide units; and
   ii) from about 10% to about 90% of a second copolymer wherein the second copolymer comprises from about 55% to about 90% D,L-lactide units and from 10% to about 45% glycolide units;
c) combining the aqueous phase from step (a) with the organic phase from step (b) to form a primary emulsion;
d) combining the primary emulsion with an aqueous continuous phase comprising from about 1.5 M to about 2.5 M NaCl to form a water/oil/water emulsion; and
e) combining the water/oil/water emulsion with an aqueous extraction phase and forming the microparticle.

A still further non-limiting example of the disclosed process for forming a microparticle of this aspect includes a process comprising:
a) providing an aqueous phase comprising from about 10 wt. % to about 30 wt. % ranibizumab and from about 0.1 wt. % to about 0.5 wt. % of poly(vinyl alcohol);
b) providing an organic phase comprising about 20 wt. % of a poly(D,L-lactide-co-glycolide) copolymer admixture comprising:
   i) from about 10% to about 90% of a first copolymer wherein the first copolymer comprises from about 65% to about 90% D,L-lactide units and from 10% to about 35% glycolide units; and
   ii) from about 10% to about 90% of a second copolymer wherein the second copolymer comprises from about 55% to about 90% D,L-lactide units and from 10% to about 45% glycolide units;
c) combining the aqueous phase from step (a) with the organic phase from step (b) to form a primary emulsion;
d) combining the primary emulsion with an aqueous continuous phase comprising from about 1.5 M to about 2.5 M NaCl and from about 1.5 wt. % to about 2.5 wt. % poly(vinyl alcohol) to form a water/oil/water emulsion; and
e) combining the water/oil/water emulsion with an aqueous extraction phase and forming the microparticle.

A still yet further non-limiting example of the disclosed process for forming a microparticle of this aspect includes a process comprising:
a) providing an aqueous phase comprising from about 10 wt. % to about 30 wt. % ranibizumab and from about 0.1 wt. % to about 0.5 wt. % of poly(vinyl alcohol);
b) providing an organic phase comprising about 20 wt. % of a poly(D,L-lactide-co-glycolide) copolymer admixture comprising:
   i) from about 10% to about 90% of a first copolymer wherein the first copolymer comprises from about 65% to about 90% D,L-lactide units and from 10% to about 35% glycolide units; and
   ii) from about 10% to about 90% of a second copolymer wherein the second copolymer comprises from about 55% to about 90% D,L-lactide units and from 10% to about 45% glycolide units;
c) combining the aqueous phase from step (a) with the organic phase from step (b) to form a primary emulsion;
d) combining the primary emulsion with an aqueous continuous phase comprising from about 1.5 M to about 2.5 M NaCl and from about 1.5 wt. % to about 2.5 wt. % poly(vinyl alcohol) to form a water/oil/water emulsion; and
e) combining the water/oil/water emulsion with an aqueous extraction phase and forming the microparticle.

Disclosed herein are methods for treating or preventing age related macular degeneration. Further disclosed are methods for treating macular degeneration and diseases, illnesses, or conditions relating to retinal edema and retinal neovascularization including increased or abnormal macular angiogenesis.

A further aspect relates to a method for treating or preventing macular degeneration comprising administering to a subject a composition comprising:

A) a microparticle comprising:
a) from about 1 wt. % to about 15 wt. % ranibizumab; and
b) a poly(D,L-lactide-co-glycolide) copolymer admixture comprising:
   i) from about 10 wt. % to about 90 wt. % of a first copolymer wherein the first copolymer comprises from about 65% to about 90% D,L-lactide units and from 10% to about 35% glycolide units; and
   ii) from about 10 wt. % to about 90 wt. % of a second copolymer wherein the second copolymer comprises from about 55% to about 90% D,L-lactide units and from 10% to about 45% glycolide units; and

B) a pharmaceutically acceptable carrier.

In one embodiment, the disclosed method relates to administering a composition comprising:

A) a microparticle comprising:
a) from about 5 wt. % to about 10 wt. % ranibizumab; and
b) a poly(D,L-lactide-co-glycolide) copolymer admixture comprising:
   i) from about 10 wt. % to about 90 wt. % of a first copolymer wherein the first copolymer comprises from about 65% to about 90% D,L-lactide units and from 10% to about 35% glycolide units; and
   ii) from about 10 wt. % to about 90 wt. % of a second copolymer wherein the second copolymer comprises from about 55% to about 90% D,L-lactide units and from 10% to about 45% glycolide units; and

B) a pharmaceutically acceptable carrier.

One example of this embodiment relates to administering compositions comprising:

A) a microparticle comprising:
a) from about 5 wt. % to about 10 wt. % ranibizumab; and
b) a poly(D,L-lactide-co-glycolide) copolymer admixture comprising:
   i) from about 10 wt. % to about 90 wt. % of a first copolymer wherein the first copolymer comprises from about 65% to about 90% D,L-lactide units and from 10% to about 35% glycolide units having an intrinsic viscosity of from about 0.4 dL/g to about 0.8 dL/g; and
   ii) from about 10 wt. % to about 90 wt. % of a second copolymer wherein the second copolymer comprises from about 55% to about 90% D,L-lactide units and from 10% to about 45% glycolide units having an intrinsic viscosity of from about 0.2 dL/g to about 0.7 dL/g; and

B) a pharmaceutically acceptable carrier.

In another embodiment, the composition comprises:

A) a microparticle comprising:
a) from about 5 wt. % to about 10 wt. % ranibizumab; and
b) a poly(D,L-lactide-co-glycolide) copolymer admixture comprising:
   i) about 50 wt. % of an ester capped poly(D,L-lactide-co-glycolide) comprising 85% D,L-lactide units and 15% glycolide units, wherein the copolymer has an intrinsic viscosity of from about 0.4 dL/g to about 0.5 dL/g; and
   ii) about 50 wt. % of an acid capped poly(D,L-lactide-co-glycolide) comprising 85% D,L-lactide units and 15% glycolide units, wherein the copolymer has an intrinsic viscosity of from about 0.55 dL/g to about 0.70 dL/g, or from about 0.55 dL/g to about 0.65 dL/g, or from about 0.60 dL/g to about 0.70 dL/g; and

B) a pharmaceutically acceptable carrier.

A further embodiment relates to a method for treating or preventing macular degeneration comprising administering to a subject a composition comprising:

A) from about 1 mg to about 500 mg of a microparticle comprising:
a) from about 1 wt. % to about 15 wt. % ranibizumab; and
b) a poly(D,L-lactide-co-glycolide) copolymer admixture comprising:
   i) about 80 wt. % of an ester capped poly(D,L-lactide-co-glycolide) comprising 75% D,L-lactide units and 25% glycolide units, wherein the copolymer has an intrinsic viscosity of from about 0.65 dL/g to about 0.75 dL/g; and
   ii) about 20 wt. % of an acid poly(D,L-lactide-co-glycolide) comprising 65% D,L-lactide units and 35% glycolide units, wherein the copolymer has an intrinsic viscosity of from about 0.35 dL/g to about 0.5 dL/g; and

B) a pharmaceutically acceptable carrier;

wherein the composition contains from about 1 wt. % to about 50 wt. % of solids.

A still further embodiment relates to a method for treating or preventing macular degeneration comprising administering to a subject a composition comprising:

A) from about 1 mg to about 500 mg of a microparticle comprising:
a) from about 1 wt. % to about 15 wt. % ranibizumab; and
b) a poly(D,L-lactide-co-glycolide) copolymer admixture comprising:
   i) about 50 wt. % of an acid capped poly(D,L-lactide-co-glycolide) comprising 85% D,L-lactide units and 15% glycolide units, wherein the copolymer has an intrinsic viscosity of from about 0.45 dL/g to about 0.55 dL/g; and
   ii) about 50 wt. % of an ester poly(D,L-lactide-co-glycolide) comprising 75% D,L-lactide units and 25% glycolide units, wherein the copolymer has an intrinsic viscosity of from about 0.45 dL/g to about 0.60 dL/g, or from about 0.45 to about 0.55 dL/g, or from about 0.50 dL/g to about 0.60 dL/g; and

B) a pharmaceutically acceptable carrier;

wherein the composition contains from about 1 wt. % to about 50 wt. % of solids.

A yet still further embodiment relates to a method for treating or preventing macular degeneration comprising administering to a subject a composition comprising:

A) from about 1 mg to about 500 mg of a microparticle comprising:
a) from about 1 wt. % to about 15 wt. % ranibizumab; and
b) a poly(D,L-lactide-co-glycolide) copolymer admixture comprising:
   i) about 80 wt. % of an acid capped poly(D,L-lactide-co-glycolide) comprising 75% D,L-lactide units and 25% glycolide units, wherein the copolymer has an intrinsic viscosity of from about 0.65 dL/g to about 0.75 dL/g; and
   ii) about 20 wt. % of an ester poly(D,L-lactide-co-glycolide) comprising 65% D,L-lactide units and 35% glycolide units,

wherein the copolymer has an intrinsic viscosity of from about 0.15 dL/g to about 0.25 dL/g; and

B) a pharmaceutically acceptable carrier;

wherein the composition contains from about 1 wt. % to about 50 wt. % of solids.

A yet still another embodiment relates to a method for treating or preventing macular degeneration comprising administering to a subject a composition comprising:

A) from about 1 mg to about 500 mg of a microparticle comprising:
a) from about 1 wt. % to about 15 wt. % ranibizumab; and
b) a poly(D,L-lactide-co-glycolide) copolymer admixture comprising:
   i) about 75 wt. % of an acid capped poly(D,L-lactide-co-glycolide) comprising 75% D,L-lactide units and 25% glycolide units, wherein the copolymer has an intrinsic viscosity of from about 0.65 dL/g to about 0.75 dL/g; and
   ii) about 25 wt. % of an ester poly(D,L-lactide-co-glycolide) comprising 65% D,L-lactide units and 35% glycolide units, wherein the copolymer has an intrinsic viscosity of from about 0.4 dL/g to about 0.5 dL/g; and

B) a pharmaceutically acceptable carrier;

wherein the composition contains from about 1 wt. % to about 50 wt. % of solids.

In all of the major aspects, the disclosed methods relate to intraocular injection directly into the vitreous humor of the eye with the microparticles or pharmaceutical compositions disclosed herein. The compositions used for the disclosed methods can have from about 10 to 500 mg, 50 to 400 mg, 50 to 300 mg, 50 to 200 mg, 50 to 150 mg, or about 100 mg of microparticles suspended in an injection vehicle. The injection vehicle, in one aspect, comprises from about 1% to about 50% solids and in another aspect from about 10% to 40% solids and in another aspect from 20% to 30% solids. In another aspect, the compositions used for the disclosed methods can have from about 10 mg to about 150 mg of microparticles suspended in an injection vehicle, wherein the injection vehicle comprises from about 20% to about 30% solids. In one aspect, the compositions can deliver from about 0.1 mg to about 3 mg of ranibizumab per dose. In another aspect the compositions can deliver from about 0.7 mg to about 1.5 mg of ranibizumab per dose. The microparticles and compositions disclosed herein are typically delivered by injecting them intravitrealy at 10 to 150 μL total volume per injection using a needle, e.g. 25-G UTW needle.

In one aspect, the dosage is a single injection each 3 to 12 months, such as, in various aspects about every 3, 6, 9 or 12 months.

In all of the major aspects the administration is to a subject or to a patient. In one specific aspect, the subject or patient is a mammal, such as a human.

In all of the major aspects, the disclosed microparticles can be formed according to a process comprising:
a) dissolving or otherwise dispersing the desired amount of ranibizumab into water to form an aqueous solution;
b) dispersing the aqueous solution of ranibizumab into a solvent containing one or more of the disclosed polymers, copolymers, or mixtures thereof, to form a primary emulsion which is referred to herein as the Dispersed Phase;
c) admixing the Dispersed Phase into an aqueous Continuous Phase thereby forming a water/oil/water emulsion (w/o/w double emulsion); and
d) diluting the w/o/w double emulsion with an aqueous Extraction Phase, thereby initiating solvent extraction and formation of microencapsulated ranibizumab.

In a further aspect, the disclosed microparticles can be formed according to a process comprising:
a) dissolving or otherwise dispersing the desired amount of ranibizumab into water to form an aqueous solution;
b) dispersing the aqueous solution of ranibizumab into a solvent containing one or more of the disclosed polymers, copolymers, or mixtures thereof, to form a primary emulsion which is referred to herein as the Dispersed Phase;
c) admixing the Dispersed Phase into an aqueous Continuous Phase, wherein the Continuous Phase comprises one or more salts, thereby forming a water/oil/water emulsion (w/o/w double emulsion); and
d) diluting the w/o/w double emulsion with an aqueous Extraction Phase, thereby initiating solvent extraction and formation of microencapsulated ranibizumab.

Any desired excipients, for example, trehalose or polysorbate 20 or combinations thereof, can be added together with the ranibizumab in step (a).

The Continuous Phase solution of step (c) may comprise a surfactant such as PVA. The Continuous Phase of step (c) may comprise salts. The Continuous Phase of step (c) may comprise the organic solvent from the Dispersed Phase at amounts up to a saturating amount in the Continuous Phase solution. Further the Continuous Phase may include a surfactant such as PVA or salts or an organic solvent at amounts up to a saturating amount in the Continuous Phase solution, or any combinations thereof.

The polymers used as the microparticle wall-forming material can be a single homopolymer, for example, poly(D,L-lactide), or a blend of two or more homopolymers and/or copolymers. When two or more polymers comprise the wall-forming material, the formulator can use any of a variety of methods known to those skilled in the art. A non-limiting example of the blending of two polymers includes the following procedure:
a) the desired amount of the polymers are charged to a suitable vessel containing an amount of one or more organic solvents;
b) the vessel is sealed (for example, stoppered) then the contents are agitated until the polymer is completely dissolved or dispersed; and
c) stored or used directly as the Dispersed Phase for forming the primary emulsion of the disclosed process.

In one specific aspect, the microparticles comprise: a) from about 1 wt. % to about 15 wt. % ranibizumab; and b) poly(D,L-lactide). The poly(D,L-lactide) can have any desirable end group, including acid or ester. The poly(D,L-lactide) can also exhibit a variety of intrinsic viscosities, for example from about 0.40 dL/g to about 0.70 dL/g, e.g., 0.40, 0.45, 0.50, 0.55, 0.60, 0.65, or 0.70 dL/g.

In one specific aspect, the disclosed microparticles comprise: a) from about 1 wt. % to about 15 wt. % ranibizumab; and b) a poly(D,L-lactide-co-glycolide) copolymer. Non-limiting examples of the poly(D,L-lactide-co-glycolide) copolymer are listed in Table A.

TABLE A
Examples of poly(D,L-lactide-co-glycolide)

| | Mole % | Mole % | | End |
|---|---|---|---|---|
| Copolymer | D,L-lactide | glycolide | Viscosity (dL/g) | Group |
| 1 | 75 | 25 | 0.55 | Ester (E) |
| 2 | 76 | 24 | 0.45 | Ester (E) |
| 3 | 77 | 23 | 0.55 | Ester (E) |
| 4 | 78 | 22 | 0.40 | Ester (E) |
| 5 | 79 | 21 | 0.55 | Ester (E) |
| 6 | 80 | 20 | 0.55 | Ester (E) |
| 7 | 81 | 19 | 0.55 | Ester (E) |
| 8 | 82 | 18 | 0.60 | Acid (A) |
| 9 | 83 | 17 | 0.40 | Acid (A) |
| 10 | 84 | 16 | 0.40 | Acid (A) |
| 11 | 85 | 15 | 0.50 | Acid (A) |
| 12 | 85 | 15 | 0.60 | Acid (A) |
| 13 | 86 | 14 | 0.45 | Acid (A) |
| 14 | 87 | 13 | 0.45 | Acid (A) |
| 15 | 88 | 12 | 0.60 | Acid (A) |
| 16 | 89 | 11 | 0.60 | Acid (A) |
| 17 | 90 | 10 | 0.60 | Acid (A) |
| 18 | 75 | 25 | 0.45 | Ester (E) |
| 19 | 76 | 24 | 0.50 | Ester (E) |
| 20 | 77 | 23 | 0.50 | Ester (E) |
| 21 | 78 | 22 | 0.45 | Ester (E) |
| 22 | 79 | 21 | 0.40 | Ester (E) |
| 23 | 80 | 20 | 0.40 | Ester (E) |
| 24 | 81 | 19 | 0.40 | Ester (E) |
| 25 | 82 | 18 | 0.50 | Acid (A) |
| 26 | 83 | 17 | 0.50 | Acid (A) |
| 27 | 84 | 16 | 0.50 | Acid (A) |
| 29 | 85 | 15 | 0.60 | Acid (A) |
| 30 | 75 | 25 | 0.50 | Acid (A) |
| 31 | 75 | 25 | 0.60 | Acid (A) |
| 32 | 75 | 25 | 0.50 | Ester (E) |
| 33 | 75 | 25 | 0.60 | Ester (E) |
| 34 | 85 | 15 | 0.45 | Acid (A) |
| 35 | 85 | 15 | 0.55 | Acid (A) |
| 34 | 85 | 15 | 0.45 | Ester (E) |
| 35 | 85 | 15 | 0.55 | Ester (E) |
| 36 | 85 | 15 | 0.70 | Acid (A) |

In another specific aspect, the disclosed microparticles comprise: a) from about 1 wt. % to about 15 wt. % ranibizumab; and b) an admixture of poly(D,L-lactide-co-glycolide) copolymers wherein the admixture comprises: i) from about 10 wt. % to about 90 wt. % of a first copolymer wherein the first copolymer comprises from about 65% to about 90% D,L-lactide units and from 10% to about 35% glycolide units; and ii) from about 10 wt. % to about 90 wt. % of a second copolymer wherein the second copolymer comprises from about 55% to about 90% D,L-lactide units and from 10% to about 45% glycolide units. For example, any two or more of the specific copolymers disclosed in Table A can be admixed together in any suitable ratio. Non-limiting examples are listed in Table B.

TABLE B

| Copolymer A^{a} | Copolymer B^{a} | wt. % Ratio A:B |
|---|---|---|
| 16 | 8 | 80:20 |
| 12 | 5 | 70:30 |
| 11 | 1 | 50:50 |
| 10 | 6 | 60:40 |
| 9 | 2 | 75:25 |
| 16 | 4 | 90:10 |
| 29 | 16 | 50:50 |
| 18 | 1 | 85:15 |
| 20 | 10 | 60:40 |
| 22 | 5 | 80:20 |
| 15 | 2 | 50:50 |

^{a}Copolymers from Table A

In yet another specific aspect, a microparticle comprises: a) from about 1 wt. % to about 15 wt. % ranibizumab; and b) a polymer admixture comprising: i) from about 60% to about 99% poly(D,L-lactide); and ii) from about 1% to about 40% polycaprolactone. Table C lists non-limiting examples of such admixtures.

TABLE C

| poly(D,L-lactide) | polycaprolactone |
|---|---|
| mole % | mole % |
| 60 | 40 |
| 65 | 35 |
| 70 | 30 |
| 75 | 25 |
| 80 | 20 |
| 85 | 15 |
| 90 | 10 |
| 95 | 5 |
| 96 | 4 |
| 97 | 3 |
| 98 | 2 |
| 99 | 1 |

The microparticles described herein can comprise any desired loading of ranibizumab, such as from about 1 wt % to about 15 wt. %. For example, ranibizumab can be present in a loading of 1 wt. %, 2 wt %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, or 15 wt. %.

In a general aspect, as discussed above, any of the above disclosed microparticles can be made by a process comprising: a) providing an aqueous phase comprising from about 10 wt. % to about 30 wt. % ranibizumab and from about 0.1 wt. % to about 0.5 wt. % of surfactant; b) providing an organic solvent and from about 10 wt. % to about 30 wt. % of polymer or polymer admixture; c) combining the aqueous phase from step (a) with the organic phase from step (b) to form a primary emulsion; d) combining the primary emulsion with an aqueous continuous phase comprising organic solvent and from about 1.5 wt. % to about 2.5 wt. % of surfactant to form a water/oil/water emulsion; and e) combining the water/oil/water emulsion with an aqueous extraction phase and forming the microparticle.

In further specific aspects, the aqueous phase in step a) can comprise 10 wt %, 12 wt. %, 13 wt. %, 15 wt. %, 18 wt. %, 20 wt. %, 22 wt. %, 25 wt. %, or 30 wt. % ranibizumab. Such an aqueous phase can also comprise 0.1 wt. %, 0.2 wt. %, 0.3 wt. %, 0.4 wt. %, or 0.5 wt. %, 0.6 wt. %, 0.7 wt. %, 0.8 wt. %, 0.9 wt. %, 1.0 wt. %, 1.1 wt. %, 1.2 wt. %, 1.3 wt. %, 1.4 wt. %, or 1.5 wt. % surfactant, such as polyvinyl alcohol. Such an aqueous phase can also comprise one or more sugars, such as Trehalose, other additives, such as amino acids, for example histadine, and other excipients, such as Tween. Such additives and excipients are typically used as a buffer for ranibizumab. Step b) above can be carried out using a variety of organic solvents, such as ethanol, methylene chloride, ethyl ether, or ethyl acetate. The polymer or polymer admixture above can be any herein disclosed polymer or polymer admixture. Step d) above can be carried out using a variety of organic solvents, such as ethanol, ethyl ether, or ethyl acetate. The aqueous continuous phase of step d) can also comprise other additives, such as salt, for example sodium chloride. An example of a suitable surfactant for step d) is polyvinyl alcohol, and such a surfactant can be present at various percentages, such as 1.5 wt. %, 2.0 wt. %, or 2.5 wt. %. Step e) above can be carried out using a variety of methods, such as stirring and filtering the combined water/oil/water emulsion with the aqueous extraction phase and subsequently drying and collecting the extracted particles. The extracted particles can be a variety of average sizes, for example, 10 or less; 15, 20, 25, 30, 40, 50, 60, 70, 80, 90, 100, 110, 125, or greater than 125 microns. In one aspect, up to 20% of the particles are less than 25 microns. In other aspects, up to 10% of the particles are less than 25 microns. In other aspects, up to 5% of the particles are less than 25 microns. For example, in one specific aspect, 5 to 10% of the particles can be less than 25 microns. In one aspect, the particles are then collected in a sieve. In one aspect, the collected sieve particles can be from 25 to 125 microns.

For various pharmaceutical formulations comprising any of the herein disclosed microparticles, the particles can be reconstituted to a viscosity range of from about 30 to about 90 cP, or from about 30 to 70 cP, or from about 30 to 50 cP, or from about 30 to 40 cP, or from about 50 to 70 cP. The reconstitution can be carried out with a variety of solutions, for example, the combination of water, tween, and phosphate buffered saline. The reconstitution formula can also comprise hyaluronic acid. Such a process can be useful for a variety of pharmaceutical formulations, such as injectable formulations. An injectable formulation can be injected into the eye to treat a variety of disorders, such as macular degeneration (MD) or diabetic macular edema (DME).

### EXAMPLES

The following examples are put forth so as to provide those of ordinary skill in the art with a complete disclosure and description of how the compounds, compositions, articles, devices, and/or methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

The following are non-limiting examples of the disclosed process. The polymers utilized in the following examples were obtained from Lakeshore Biomaterials, Birmingham, Ala. The following designation is used herein to describe the polymers and copolymers used to form the disclosed microparticles:

| | | | | |
|---|---|---|---|---|
| 85 | 15 | DLG | 4 | E |

wherein 85 is the mole % of a first monomer, and 15 is the mole % of the second monomer. A polymer comprising 100% lactide units, i.e., poly(D,L-lactide) will be represented by “100DL.” Polymer abbreviations used herein are DLG represents poly(D,L-lactide-co-glycolide), DL represents poly(D,L-lactide) and CL represents polycaprolactone. The next integer represents a target intrinsic viscosity, IV value. For example, the number 4 represents 0.4 dL/g and the number 6.5 represents 0.65 dL/g. The final letter designates whether the polymer has an end group that is and ester (E) or an acid (A).

### Example 1

Microparticle Example A: A polymer solution was prepared by dissolving 8515 DLG 4.5E (8.5 g) in ethyl acetate (34 g). The resulting polymer solution contained 20 wt % polymer. In a separate vessel, lyophilized ranibizumab (1.5 g) and trehalose (1.0 g) were dissolved in an aqueous solution containing 0.5 wt % of poly(vinyl alcohol) (6.8 g). The aqueous solution containing ranibizumab and the ethyl acetate solution containing the polymer were combined at room temperature and formed into an emulsion (Dispersed Phase) using an IKA Ultra Turrax T25 Basic homogenizer. A second Continuous Phase solution was prepared by adding ethyl acetate (15.6 g) and sodium chloride (54 g) to an aqueous solution containing 2 wt % of poly(vinyl alcohol) (465 g). The poly(vinyl alcohol) used in this example is available from Amresco; Solon, Ohio. The Dispersed Phase emulsion was then emulsified into the second Continuous Phase solution at room temperature using a SILVERSON™ L4R continuous mixer. The resulting emulsion was then immediately added to a tank containing approximately 2 L of an aqueous Extraction Phase of dionized water and the admixture stirred with a magnetic stirrer.

After approximately 30 minutes of extraction time, the resulting suspension was passed through two 8-inch diameter test sieves where the first sieve had a mesh size of 125 microns and the second sieve had a mesh size of 25 microns (RETSCH™ or FISHER™ test sieves). The microparticle product material that passed through the 125 micron sieve but that was collected on top of the 25 micron test sieve was then rinsed with 2 L of deionized water. This product was then dried with minimal airflow at room temperature. After drying, the microparticle product was transferred to a polypropylene vial and stored refrigerated. The results are shown below in Table I, Example 1A. Microparticles corresponding to Examples 1B-F were prepared in a similar manner to the microparticles formed in Examples 1A and the results are provided in Table I below.

### Example 2

Microparticle Example G: A polymer solution was prepared by dissolving 100 DL 5E (8.55 g) and 100 CL 10E (0.45 g) in dichloromethane (27 g). The resulting polymer solution contained 25 wt % polymer. In a separate vessel, lyophilized ranibizumab (1.0 g) and trehalose (0.67 g) were dissolved in an aqueous solution containing 0.1 wt % of poly(vinyl alcohol) (4.5 g). The aqueous solution containing ranibizumab and the ethyl acetate solution containing the polymer were combined at room temperature and formed into an emulsion (Dispersed Phase) using an IKA Ultra Turrax T25 Basic homogenizer. A second Continuous Phase solution was prepared by adding sodium chloride (44 g) to an aqueous solution containing 2 wt % of poly(vinyl alcohol) (380 g). The poly(vinyl alcohol) used in this example is available from Amresco; Solon, Ohio. The Dispersed Phase emulsion was then emulsified into the second Continuous Phase solution at room temperature using a SILVERSON™ L4R continuous mixer. The resulting emulsion was then immediately added to a tank containing approximately 5 L of an aqueous Extraction Phase of dionized water and the admixture stirred with a magnetic stirrer.

After approximately 30 minutes of extraction time, the resulting suspension was passed through two 8-inch diameter test sieves where the first sieve had a mesh size of 125 microns and the second sieve had a mesh size of 20 microns (RETSCH™ or FISHER™ test sieves). The microparticle product material that passed through the 125 micron sieve but that was collected on top of the 25 micron test sieve was then rinsed with 2 L of deionized water. This product was then dried with minimal airflow at room temperature. After drying, the microparticle product was transferred to a polypropylene vial and stored refrigerated. The results are shown below in Table I, Example 2G. Microparticles corresponding to Examples 2H-K were prepared according to Example 2G. The results are contained in Table I below.

### Example 3

Microparticle Example H: A polymer solution was prepared by dissolving 100 DL 5E (4.5 g) in dichloromethane (13.5 g). The resulting polymer solution contained 25 wt % polymer. In a separate vessel, lyophilized ranibizumab (0.5 g) and trehalose (0.33 g) were dissolved in an aqueous solution containing 0.1 wt % of poly(vinyl alcohol) (2.25 g). The aqueous solution containing ranibizumab and the ethyl acetate solution containing the polymer were combined at room temperature and formed into an emulsion (Dispersed Phase) using an IKA Ultra Turrax T25 Basic homogenizer. A second Continuous Phase solution was prepared by adding dichloromethane (7 g) to an aqueous solution containing 2 wt % of poly(vinyl alcohol) (314 g). The poly(vinyl alcohol) used in this example is available from Amresco; Solon, Ohio. The Dispersed Phase emulsion was then emulsified into the second Continuous Phase solution at room temperature using a SILVERSON™ L4R-TA probe mixer for 45 seconds. The resulting emulsion was then immediately added to a tank containing approximately 3 to 4 L of an aqueous Extraction Phase of dionized water and the admixture stirred with a magnetic stirrer.

After approximately 30 minutes of extraction time, the resulting suspension was passed through two 8-inch diameter test sieves where the first sieve had a mesh size of 125 microns and the second sieve had a mesh size of 20 microns (RETSCH™ or FISHER™ test sieves). The microparticle product material that passed through the 125 micron sieve but that was collected on top of the 25 micron test sieve was then rinsed with 2 L of deionized water. This product was then dried with minimal airflow at room temperature. After drying, the microparticle product was transferred to a polypropylene vial and stored refrigerated.

TABLE I

| | Polymer 1 | | Polymer 2 | |
|---|---|---|---|---|
| Example | | Quantity, | | Quantity, |
| number | Description | g | Description | g |
| 1A | 8515 DLG 4.5E | 8.5 | — | — |
| 1B | 8515 DLG 7A | 8.5 | — | — |
| 1C | 8515 DLG 4.5E | 4.25 | 8515 DLG 7A | 4.25 |
| 1D | 8515 DLG 5A | 6.375 | 7525 DLG 5.5E | 2.125 |
| 1E | 7525 DLG 7A | 6.375 | 6535 DLG 2E | 2.125 |
| 1F | 7525 DLG 7E | 6.375 | 6535 DLG 4.5A | 2.125 |
| 2G | 100 DL 5E | 8.55 | 100 CL 10E | 0.45 |
| 2H | 100 DL 5E | 9.0 | — | — |
| 2J | 100 DL 5E | 9.0 | — | — |
| 2K | 8515 DLG 4.5E | 9.0 | — | — |

### Example 4

Examples of the microparticles formed by the disclosed methods were evaluated for the amount of ranibizumab loaded into the microparticles. The amount of active was evaluated by SEC and IEC. The results are shown in Table II.

TABLE II

| | | ranibizumab | | |
|---|---|---|---|---|
| | | content | ranibizumab content | loading |
| No. | polymer | (SEC) wt. % | (IEC) wt. % | efficiency |
| G | 95% 100 DL 5E | 7.0 | 6.6 | 71.0 |
| | 5% 100 CL 10E | | | |
| H | 100 DL 5E | 5.2 | 4.9 | 62.0 |
| J | 100 DL 3E | 6.1 | 6.1 | 76.3 |
| K | 8515 DLG 4.5E | 7.0 | 6.3 | 87.5 |
| L | 100 DL 5E | 5.1 | 5.3 | 74.6 |
| M | 8515 DLG 7A | 4.9 | 4.4 | 63.0 |

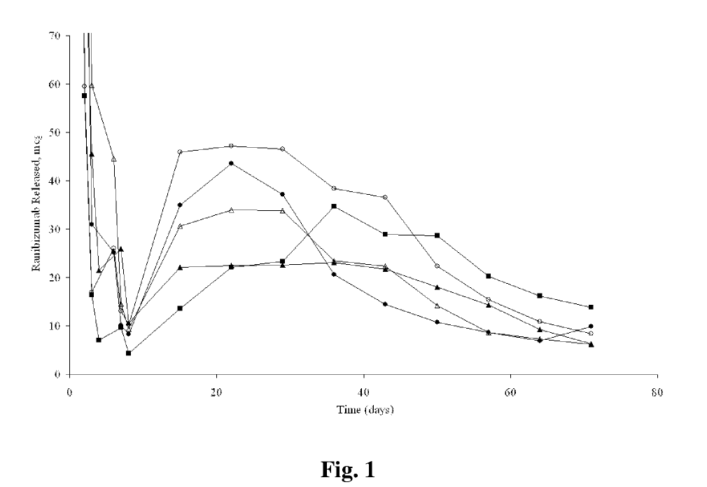

FIG. 1 depicts the in vitro elution profiles of ranibizumab microspheres in 100 mM PBS/0.5% BSA/0.05% Proclin 300 at 37° C. The line having solid triangles (▴) corresponds to a polymer blend of 8515 DLG 4.5E and 8515 DLG 6A; the line having clear triangles (Δ) corresponds to a polymer blend of 8515 DLG 5A and 7525 DLG 5.5E; the line having solid circles () corresponds to a polymer blend of 7525 DLG 7A and 6535 DLG 2E; the line having clear circles (◯) corresponds to a polymer blend of 7525 DLG 7E and 6535 DLG 4.5A; and the line having solid squares (▪) corresponds to copolymer 8515 DLG 7A.

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

What is claimed is:

1. A pharmaceutical composition comprising a microparticle comprising:
a) from about 1 wt. % to about 15 wt. % ranibizumab; and
b) poly(D,L-lactide-co-glycolide) copolymer wherein the copolymer comprises:
i) from about 75% to about 90% D,L-lactide units; and
ii) from about 10% to about 25% glycolide units.

2. The composition according to claim 1, wherein the composition comprises from about 10 mg to about 500 mg of the microparticles.

3. The composition according to claim 1, wherein the composition contains from about 10 wt. % to about 40 wt. % of solids.

4. The composition according to claim 1, wherein the copolymer has an intrinsic viscosity of from about 0.2 dL/g to about 0.8 dL/g.

5. The composition according to claim 1, wherein the microparticle comprises from about 5 wt. % to about 10 wt. % of ranibizumab.

6. The composition according to claim 1, wherein the microparticle comprises one or more pharmaceutically acceptable excipients.

7. The composition according to claim 1, wherein the microparticle further comprises trehalose.

8. The composition according to claim 1, wherein the microparticle releases ranibizumab over a period at least about 3 months.

9. The composition according to claim 1, wherein the microparticle releases ranibizumab over a period of at least about 6 months.

10. A microparticle comprising ranibizumab in an amount of from 1 to 15 wt. % of the microparticle and a poly(lactide-co-glycolide) copolymer having from 75 wt. % to 90 wt. % lactide units and from 25 wt. % to 10 wt. % glycolide units, in the form of a microparticle.

11. A method of preventing or treating age-related macular degeneration in a subject comprising administering to a subject in need of such treatment an effective amount of the composition according to claim 1.

12. A pharmaceutical composition comprising a microparticle comprising:
a) from about 1 wt. % to about 15 wt. % ranibizumab; and
b) an admixture of poly(D,L-lactide-co-glycolide) copolymers wherein the admixture comprises:
i) from about 10 wt. % to about 90 wt. % of a first copolymer wherein the first copolymer comprises from about 65% to about 90% D,L-lactide units and from 10% to about 35% glycolide units; and
ii) from about 10 wt. % to about 90 wt. % of a second copolymer wherein the second copolymer comprises from about 55% to about 90% D,L-lactide units and from 10% to about 45% glycolide units.

13. The composition according to claim 12, wherein the first copolymer comprises:
i) from about 75% to about 85% D,L-lactide units; and
ii) from about 15% to about 25% glycolide units; and
the second copolymer comprises:
i) from about 65% to about 85% D,L-lactide units; and
ii) from about 15% to about 35% glycolide units.

14. The composition according to claim 12, wherein the first copolymer is from about 45 wt. % to about 80 wt. % and the second copolymer is from about 20 wt. % to about 55 wt. % of the copolymer admixture.

15. The composition according to claim 12, wherein the copolymer admixture has an intrinsic viscosity of from about 0.2 dL/g to about 0.8 dL/g.

16. The composition according to claim 12, wherein the microparticle comprises from about 7 wt. % to about 10 wt. % of ranibizumab.

17. The composition according to claim 12, wherein the microparticle further comprises trehalose.

18. The composition according to claim 12, wherein the microparticle releases ranibizumab over a period of at least about 6 months.

19. A microparticle comprising ranibizumab in an amount of from 1 to 15 wt. % of the microparticle and an admixture of poly(D,L-lactide-co-glycolide) copolymers wherein the admixture comprises
a) from about 10% to about 90% of a first copolymer wherein the first copolymer comprises from about 65% to about 90% D,L-lactide units and from 10% to about 35% glycolide units; and
b) from about 10 wt. % to about 90 wt. % of a second copolymer wherein the second copolymer comprises from about 55% to about 90% D,L-lactide units and from 10% to about 45% glycolide units.

20. A method of preventing or treating age-related macular degeneration in a subject comprising administering to a subject in need of such treatment an effective amount of a composition according to claim 12.
